(19) United States
(12) Patent Application Publication
(10) Pub. No.: US 2022/0296159 A1
(43) Pub. Date: Sep. 22, 2022

(54) SLEEP EVALUATION METHOD, SLEEP EVALUATION SYSTEM, TERMINAL DEVICE, AND RECORDING MEDIUM

(71) Applicant: Panasonic Intellectual Property Management Co., Ltd., Osaka (JP)

(72) Inventors: Etsuko MIZUNO, Osaka (JP); Taiji SASAKI, Osaka (JP); Yasuo KOHASHI, Osaka (JP); Masashi SUGIYAMA, Osaka (JP)

(21) Appl. No.: 17/640,123

(22) PCT Filed: Feb. 19, 2021
(86) PCT No.: PCT/JP2021/006426

§ 371 Date: Mar. 3, 2022

(30) Foreign Application Priority Data
Feb. 21, 2020 (JP) 2020-027811
Feb. 19, 2021 (JP) 2021-024781

(51) Int. Cl. A61B 5/00; G16H 20/00; G16H 10/60

(52) U.S. Cl. CPC A61B 5/4815; A61B 5/7475; A61B 5/742; A61B 5/0022; G16H 20/00; G16H 10/60

(57) ABSTRACT

Weight information is determined for each of an individual evaluation criterion that is determined on the basis of a sleep log that includes a log of specific sleep information relating to the sleep of a specific user and a general evaluation criterion that is determined on the basis of sleep information relating to the sleep of a plurality of users; and an evaluation process is performed, which is a process of evaluating the sleep of the specific user from the specific sleep information, using the individual evaluation criterion, the general evaluation criterion, and the weight information.

### CROSS-REFERENCE OF RELATED APPLICATIONS

This application is the U.S. National Phase under 35 U.S.C. § 371 of International Patent Application No. PCT/JP2021/006426, filed on Feb. 19, 2021, which in turn claims the benefit of Japanese Application No. 2020-027811, filed on Feb. 21, 2020, and Japanese Application No. 2021-024781, filed on Feb. 19, 2021, the entire disclosures of which Applications are incorporated by reference herein.

### TECHNICAL FIELD

The present disclosure generally relates to a sleep evaluation method, a sleep evaluation system, a terminal device, and a recording medium, and more particularly relates to a sleep evaluation method, a sleep evaluation system, a terminal device, and a recording medium for evaluating the sleep of a specific user.

### BACKGROUND ART

Patent Literature (PTL) 1 discloses a sleep state determination device (sleep evaluation system) that determines a user's sleep state. The sleep state determination device disclosed in PTL 1 includes a first data acquisition part that acquires vital data of the user sleeping and a second data acquisition part that acquires the user's subjective data relating to sleep. The sleep state determination device disclosed in PTL 1 includes a first judgement part that judges the user's sleep state on the basis of the data acquired by the first and second data acquisition parts.

### CITATION LIST

### Patent Literature

[PTL 1] Japanese Patent No. 6555696

### SUMMARY OF INVENTION

### Technical Problem

In PTL 1, a sleep state is determined on the basis of the user's subjective view. To perform an appropriate sleep evaluation, however, the use of the user's subjective view is not enough.

It is thus an aim to provide a sleep evaluation method, a sleep evaluation system, a terminal device, and a recording medium capable of improving the appropriateness of a sleep evaluation.

### Solution to Problem

An aspect according to the present disclosure is a sleep evaluation method that includes: determining weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and performing an evaluation process of evaluating the sleep of the specific user from the specific sleep information, using the individual evaluation criterion, the general evaluation criterion, and the weight information.

An aspect according to the present disclosure is a sleep evaluation system that includes: a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and an evaluator that performs an evaluation process of evaluating the sleep of the specific user from the specific sleep information, using the individual evaluation criterion, the general evaluation criterion, and the weight information.

An aspect according to the present disclosure is a sleep evaluation system that includes: a server; and a terminal device capable of communicating with the server. The server includes: a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and an evaluator that performs an evaluation process of evaluating the sleep of the specific user from the specific sleep information, using the individual evaluation criterion, the general evaluation criterion, and the weight information. The terminal device includes: a communicator capable of communicating with the server; an output unit that outputs information of the terminal device; and a processing unit that controls the communicator and the output unit, and the processing unit receives via the communicator a result of the evaluation process from the evaluator of the server, and outputs via the output unit the result of the evaluation process.

An aspect according to the present disclosure is a terminal device capable of communicating with a server that includes: a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and an evaluator that performs an evaluation process of evaluating the sleep of the specific user from the specific sleep information, using the individual evaluation criterion, the general evaluation criterion, and the weight information. The terminal device includes: a communicator capable of communicating with the server; an output unit that outputs information of the terminal device; and a processing unit that controls the communicator and the output unit, wherein the processing unit receives via the communicator a result of the evaluation process from the evaluator of the server, and outputs via the output unit the result of the evaluation process.

An aspect according to the present disclosure is a non-transitory computer-readable recording medium having a program recorded thereon for causing at least one processor to execute a sleep evaluation method that includes: determining weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and performing an evaluation process of evaluating the sleep of the specific user from the specific sleep information, using the individual evaluation criterion, the general evaluation criterion, and the weight information.

An aspect according to the present disclosure is a non-transitory computer-readable recording medium having a program recorded thereon for causing at least one processor in a processing unit of a terminal device in a sleep evaluation system that includes: a server; and a terminal device capable of communicating with the server. Here, the server includes: a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and an evaluator that performs an evaluation process of evaluating the sleep of the specific user from the specific sleep information, using the individual evaluation criterion, the general evaluation criterion, and the weight information. Here, the terminal device includes: a communicator capable of communicating with the server; an output unit that outputs information of the terminal device; and the processing unit that controls the communicator and the output unit. The processing unit receives via the communicator a result of the evaluation process from the evaluator of the server, and outputs via the output unit the result of the evaluation process. The program causes the at least one processor to execute: receiving via the communicator the result of the evaluation process from the evaluator of the server; and outputting via the output unit the result of the evaluation process.

### Advantageous Effects of Invention

According to the aspects of the present disclosure, it is possible to provide the effects of improving the appropriateness of a sleep evaluation.

### BRIEF DESCRIPTION OF DRAWINGS

FIG. 1 is a diagram for explaining a sleep evaluation system according to an embodiment.

FIG. 2 is a block diagram of the sleep evaluation system.

FIG. 3 is a block diagram of a terminal device that is used together with the sleep evaluation system.

FIG. 4 is a diagram for explaining an exemplary result display screen in the sleep evaluation system.

FIG. 5 is a diagram for explaining an exemplary result display screen in the sleep evaluation system.

FIG. 6 is a flowchart of an exemplary operation performed by the sleep evaluation system.

FIG. 7 is a flowchart of an exemplary operation performed by the sleep evaluation system.

FIG. 8 is a flowchart of an exemplary operation performed by the sleep evaluation system.

FIG. 9 is a flowchart of an exemplary operation performed by the sleep evaluation system.

FIG. 10 is a diagram showing a graph representing an exemplary ratio of the weight of an individual evaluation criterion to the number of days of accumulation.

FIG. 11 is a flowchart of an exemplary operation performed by the sleep evaluation system.

FIG. 12 is a diagram showing a graph representing an exemplary ratio of the weight of the individual evaluation criterion to the number of days of accumulation.

FIG. 13 is a flowchart of an exemplary operation performed by the sleep evaluation system.

FIG. 14 is a flowchart of an exemplary operation performed by the sleep evaluation system.

FIG. 15 is a flowchart of an exemplary operation performed by the sleep evaluation system.

FIG. 16 is a diagram for explaining an exemplary result display screen in the sleep evaluation system.

FIG. 17 is a diagram for explaining an exemplary result display screen in the sleep evaluation system.

FIG. 18 is a diagram for explaining an exemplary result display screen in the sleep evaluation system.

FIG. 19 is a flowchart of an exemplary operation performed by the sleep evaluation system.

FIG. 20 is a diagram for explaining an exemplary individual evaluation criterion before and after being changed.

### DESCRIPTION OF EMBODIMENT

### (1) Embodiment

(1-1) Overview

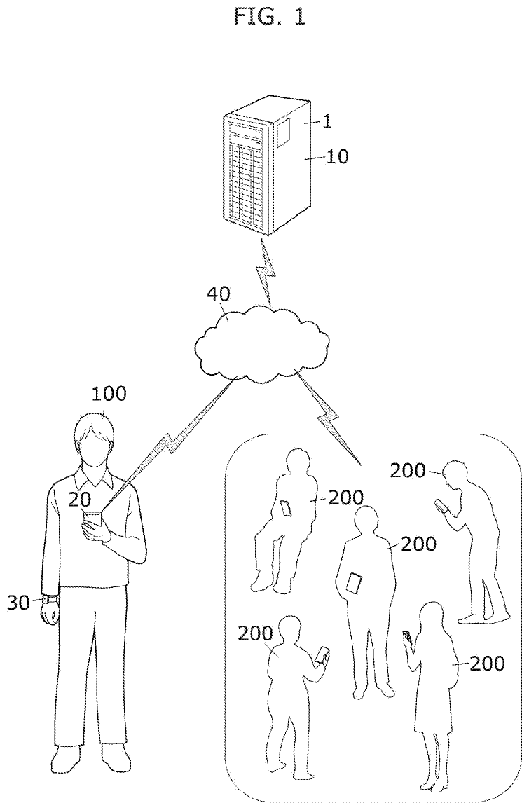

FIG. 1 is a diagram for explaining sleep evaluation system 1 according to an embodiment. Sleep evaluation system 1 is a system for evaluating the sleep of specific user 100. In the present embodiment, specific user 100 is one of a plurality of users 200.

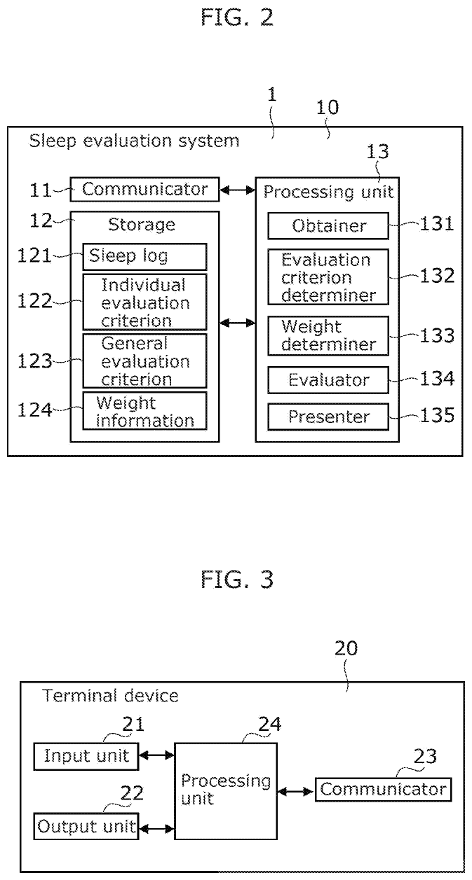

As shown in FIG. 2, sleep evaluation system 1 includes evaluation criterion determiner 132 and evaluator 134. Evaluation criterion determiner 132 determines individual evaluation criterion 122 on the basis of sleep log 121 that includes a log of specific sleep information relating to the sleep of specific user 100. Evaluator 134 performs an evaluation process of evaluating the sleep of specific user 100 from the specific sleep information, using both individual evaluation criterion 122 and general evaluation criterion 123 that is determined on the basis of sleep information relating to the sleep of a plurality of users 200.

Sleep evaluation system 1 according to the present embodiment evaluates the sleep of specific user 100 using both individual evaluation criterion 122 and general evaluation criterion 123. This enables sleep evaluation system 1 to perform an evaluation that maintains an objective standpoint, compared to the case of performing an evaluation using only individual evaluation criterion 122. Further, sleep evaluation system 1 is capable of performing an evaluation suited to specific user 100 compared to the case of performing an evaluation using only general evaluation criterion 123. As described above, sleep evaluation system 1 is capable of evaluating the sleep of specific user 100 in consideration of a comparison of the result of an evaluation that uses individual evaluation criterion 122 and the result of an evaluation that uses general evaluation criterion 123. Sleep evaluation system 1 is thus capable of improving the appropriateness of a sleep evaluation. In particular, the use of individual evaluation criterion 122 facilitates the agreement of the evaluation result to what specific user 100 actually senses. It is thus expected to increase the level of satisfaction of specific user 100 with a sleep evaluation performed by sleep evaluation system 1. Meanwhile, the use of general evaluation criterion 123 enables a sleep evaluation that takes into consideration an ideal, academically preferable sleep state obtained from the sleep information of a plurality of users 200. As a result, the effect of sleep improvement is expected.

(1-2) Details

The following describes in greater detail sleep evaluation system 1 according to the present embodiment with reference to the drawings.

Sleep evaluation system 1 is connectable to terminal device 20 via communication network 40. Note that communication network 40 can include the Internet. Communication network 40 can include not only a network compliant with a single communication protocol, but also a plurality of networks compliant with different communication protocols. The communication protocol(s) can be selected from a variety of known wired and wireless communication standards. Although simplified in FIG. 1, the communication network can include data communication devices such as a repeater hub, a switching hub, a bridge, a gateway, a router, etc.

Terminal device 20 is an information terminal, an example of which is a smartphone. Note that terminal device 20 is not limited to a smartphone, and thus may be, for example: a mobile information terminal such as a tablet terminal; a personal computer such as a desktop computer and a laptop computer; or a smart television, etc.

As shown in FIG. 3, terminal device 20 includes input unit 21, output unit 22, communicator 23, and processing unit 24.

Input unit 21 is used to input information to terminal device 20. Input unit 21 includes: an input device for operating terminal device 20; a sound input device; and a camera. The input device includes, for example, a touchpad and/or at least one button. The input device is not limited to a touchpad, and thus may be, for example, a keyboard, a pointing device, a mechanical switch, etc. Input unit 21 may include a plurality of input devices.

Output unit 22 is used to output information from terminal device 20. Output unit 22 includes an output device for presenting information viewed in terminal device 20. The output device includes an image display device for displaying information viewed in terminal device 20. The image display device is a low-profile display device such as a liquid-crystal display and an organic EL display. Note that the touchpad of input unit 21 and the image display device of output unit 22 may form a touch panel. The output device may include a sound output device that outputs information viewed in terminal device 20 in the form of sound.

Communicator 23 is connectable to communication network 40 and capable of performing communications via communication network 40. Communicator 23 is capable of communicating with server 10. Communicator 23 is compliant with a predetermined communication protocol. The predetermined communication protocol can be selected from a variety of known wired and wireless communication standards.

Processing unit 24 can be implemented by, for example, a computer system including at least one processor (microprocessor) and at least one memory. Processing unit 24 can be implemented as, for example, a computer system including at least one processor (microprocessor) and at least one memory. Stated differently, at least one processor executes at least one (computer) program (application) stored in at least one memory, thereby serving as processing unit 24. Here, the program is preliminarily stored in the memory of processing unit 24, but may be provided via a telecommunications line such as the Internet. Alternatively, the program may be recorded and provided in a non-transitory recording medium such as a memory card.

Processing unit 24 is configured to perform an overall control of terminal device 20. Stated differently, processing unit 24 is configured to control input unit 21, output unit 22, and communicator 23. Processing unit 24 is capable of sending information inputted in accordance with an operation performed in input unit 21 from communicator 23 to sleep evaluation system 1 via communication network 40. Processing unit 24 is capable of receiving information (the result of an evaluation process performed by evaluator 134) from sleep evaluation system 1 via communicator 23 over communication network 40. Processing unit 24 is capable of outputting, from output unit 22, information (the result of an evaluation process performed by evaluator 134) from sleep evaluation system 1. As described above, processing unit 24 receives, via communicator 23, the result of an evaluation process from evaluator 134 of server 10 and outputs, via output unit 22, the result of the evaluation process. The function of processing unit 24 is implemented by at least one processor of processing unit 24 executing the program.

In the present embodiment, terminal device 20 is capable of communicating with sensor 30 shown in FIG. 1. Sensor 30 is used to obtain sleep information relating to the sleep of users 200 (including specific user 100). Here, the sleep information of specific user 100 is referred to as specific sleep information. Sensor 30 is a sensor known as a sleep gauge. Examples of the sleep gauge include a stationary sleep gauge, a mattress sleep gauge, and a wearable sleep gauge. Sensor 30 in the present embodiment is a wearable sleep gauge. In the present embodiment, terminal device 20 communicates with sensor 30, thereby obtaining sleep information from sensor 30. With this, it is possible for specific user 100 to check his/her own sleep information, using terminal device 20. The sleep information includes at least one sleep-related item. In the present embodiment, the sleep information includes a plurality of sleep-related items. The sleep-related items are sleep duration, sleep efficiency, sleep latency (time taken to fall asleep), the number of mid-sleep awakenings, the state immediately before awakening, and the ratio of deep sleep. Note that a conventionally known sleep gauge can be utilized as sensor 30, and thus sensor 30 will not be described in detail.

As shown in FIG. 2, sleep evaluation system 1 includes communicator 11, storage 12, and processing unit 13. Sleep evaluation system 1 can be implemented by, for example, server 10.

Communicator 11 is a communication interface. Communicator 11 is connectable to communication network 40 and capable of performing communications via communication network 40. Communicator 11 is compliant with a predetermined communication protocol. The predetermined communication protocol can be selected from a variety of known wired and wireless communication standards. Communicator 11 is connected to terminal device 20 to be able to communicate with terminal device 20. This enables sleep evaluation system 1 to communicate with terminal device 20.

Storage 12 is used to store information used by processing unit 13 and information generated by processing unit 13. For example, storage 12 stores sleep log 121, individual evaluation criterion 122, general sleep criterion 123, and weight information 124. Storage 12 includes at least one storage device. Examples of the storage device include a random access memory (RAM) and electrically erasable programmable read only memory (EEPROM).

General evaluation criterion 123 is determined on the basis of the sleep information relating to the sleep of a plurality of users 200. General evaluation criterion 123 includes evaluation conditions for determining evaluation values relating to sleep. The evaluation conditions are set on the basis of the sleep information relating to the sleep of a plurality of users 200. Here, the evaluation conditions are set such that the respective evaluation values are greater for sleep with higher quality. In the present embodiment, evaluation values are represented as points. The result of a sleep evaluation that uses general evaluation criterion 123 is represented as a score between 1 point and 100 points.

General evaluation criterion 123 includes an evaluation criterion for each of the sleep-related items. An evaluation condition is set on an evaluation criterion basis. General evaluation criterion 123 includes an evaluation condition on an evaluation criterion basis. In the present embodiment, sleep information includes sleep duration, sleep efficiency, sleep latency (time taken to fall asleep), the number of mid-sleep awakenings, the state immediately before awakening, and the ratio of deep sleep. The sleep-related items correspond to sleep duration, sleep efficiency, sleep latency (time taken to fall asleep), the number of mid-sleep awakenings, the state immediately before awakening, and the ratio of deep sleep. General evaluation criterion 123 includes an evaluation condition for sleep duration, an evaluation condition for sleep efficiency, an evaluation condition for sleep latency, an evaluation condition for the number of mid-sleep awakenings, an evaluation condition for the state immediately before awakening, and an evaluation condition for the ratio of deep sleep. The result of a sleep evaluation that uses general evaluation criterion 123 is a total of: an evaluation value based on the evaluation condition for sleep duration (sleep duration evaluation value) Vg1; an evaluation value based on the evaluation condition for sleep efficiency (sleep efficiency evaluation value) Vg2; an evaluation value based on the evaluation condition for sleep latency (sleep latency evaluation value) Vg3; an evaluation value based on the evaluation condition for the number of mid-sleep awakenings (mid-sleep awakening evaluation value) Vg4; an evaluation value based on the evaluation condition for the state immediately before awakening (state immediately before awakening evaluation value) Vg5; and an evaluation value based on the evaluation condition for the ratio of deep sleep (deep sleep ratio evaluation value) Vg6. Thus, supposing that the result of a sleep evaluation that uses general evaluation criterion 123 is Vg, it is written as Vg=ΣVgn, where n is equal to 1 through 6.

The evaluation condition for sleep duration is a condition for converting sleep duration into an evaluation value. The evaluation condition for sleep duration is set so that the evaluation value is greater for sleep duration within an appropriate range and so that the evaluation value is smaller for sleep duration less than or longer than the appropriate range. In the present embodiment, the evaluation value for sleep duration is assigned 30 points out of 100 points. For example, under the evaluation condition for sleep duration, the evaluation value is 30 points for sleep duration between 6 hours and 9 hours, inclusive. The evaluation value is 0 points for sleep duration of 3 hours or less or 12 hours or more. When sleep duration is within a range between 3 hours and 6 hours, the evaluation value monotonously increases from 0 points to 30 points with an increase in the sleep duration. When sleep duration is within a range between 9 hours and 12 hours, the evaluation value monotonously decreases from 30 points to 0 points with an increase in the sleep duration.

The evaluation condition for sleep efficiency is a condition for converting sleep efficiency into an evaluation value. The evaluation condition for sleep efficiency is set so that the evaluation value is greater for a higher sleep efficiency. In the present embodiment, the evaluation value for sleep efficiency is assigned 30 points out of 100 points. For example, under the evaluation condition for sleep efficiency, the evaluation value is 0 points for sleep efficiency between 0% and 50%, inclusive. When sleep efficiency is within a range between 50% and 95%, the evaluation value monotonously increases from 0 points to 30 points with an increase in the sleep efficiency. When sleep efficiency is between 95% and 100%, inclusive, the evaluation value is 30 points.

The evaluation condition for sleep latency is a condition for converting sleep latency into an evaluation value. The evaluation condition for sleep latency is set so that the evaluation value is greater for sleep latency within an appropriate range, and so that the evaluation value is smaller for sleep latency less than or longer than the appropriate range. In the present embodiment, the evaluation value for sleep latency is assigned 10 points out of 100 points. For example, under the evaluation condition for sleep latency, the evaluation value is 10 points for sleep latency between 10 minutes and 30 minutes, inclusive. The evaluation value is 0 points for sleep latency of 60 minutes or longer. When sleep latency is within a range between 0 minutes and 10 minutes, the evaluation value monotonously increases from 5 points to 10 points with an increase in the sleep latency. When sleep latency is within a range between 30 minutes and 60 minutes, the evaluation value monotonously decreases from 10 points to 0 points with an increase in the sleep latency.

The evaluation condition for the number of mid-sleep awakenings is a condition for converting the number of mid-sleep awakenings into an evaluation value. The evaluation condition for the number of mid-sleep awakenings is set so that the evaluation value is greater for a smaller number of mid-sleep awakenings. In the present embodiment, the evaluation value for the number of mid-sleep awakenings is assigned 10 points out of 100 points. For example, under the evaluation condition for the number of mid-sleep awakenings, the evaluation value is 10 points for the number of mid-sleep awakenings of 0 times. The evaluation value is 7 points for the number of mid-sleep awakenings of once. The evaluation value is 5 points for the number of mid-sleep awakenings of twice. The evaluation value is 0 points for the number of mid-sleep awakenings of 3 times or more.

The evaluation condition for the state immediately before awakening is a condition for converting the state immediately before awakening into an evaluation value. Under the evaluation condition for the state immediately before awakening, the evaluation value is set in accordance with the state immediately before awakening. The state immediately before awakening refers to a sleep stage at the time of awakening. Sleep stages include a rapid eye movement period (REM sleep period) and a non-rapid eye movement period (non-REM sleep period). The non-REM period (non-REM sleep period) is divided into a light sleep period and a deep sleep period. In the present embodiment, the state immediately before awakening includes the REM sleep period, the light sleep period, the deep sleep period, and an anomaly value. The anomaly value corresponds to the case where none of the REM sleep period, the light sleep period, and the deep sleep period is applicable. In the present embodiment, the evaluation value for the state immediately before awakening is assigned 10 points out of 100 points. For example, under the evaluation condition for the state immediately before awakening, the evaluation value is 5 points when the state immediately before awakening is in the REM sleep period. The evaluation value is 10 points when the state immediately before awakening is in the light sleep period. The evaluation value is 0 points when the state immediately before awakening is in the deep sleep period. The evaluation value is 5 points when the state immediately before awakening corresponds to an anomaly value.

The evaluation condition for the ratio of deep sleep is a condition for converting the ratio of deep sleep into an evaluation value. The evaluation condition for the ratio of deep sleep is set so that the evaluation value is greater for a higher ratio of deep sleep. In the present embodiment, the evaluation value for the ratio of deep sleep is assigned 10 points out of 100 points. For example, under the evaluation condition for the ratio of deep sleep, the evaluation value is 0 points for the ratio of deep sleep between 0% and 30%, inclusive. When the ratio of deep sleep is within a range between 30% and 50%, the evaluation value monotonously increases from 0 points to 10 points with an increase in the ratio of deep sleep. When the ratio of deep sleep is between 50% and 100%, inclusive, the evaluation value is 10 points.

Processing unit 13 is configured to perform an overall control of sleep evaluation system 1. Stated differently, processing unit 13 is configured to control communicator 11 and storage 12. Processing unit 13 can be implemented by, for example, a computer system including at least one processor (microprocessor) and at least one memory. Stated differently, at least one processor executes at least one (computer) program (application) stored in at least one memory, thereby serving as processing unit 13. Here, the program is preliminarily stored in the memory of processing unit 13, but may be provided via a telecommunications line such as the Internet. Alternatively, the program may be recorded and provided in a non-transitory recording medium such as a memory card.

As shown in FIG. 2, processing unit 13 includes obtainer 131, evaluation criterion determiner 132, weight determiner 133, evaluator 134, and presenter 135. In FIG. 2, obtainer 131, evaluation criterion determiner 132, weight determiner 133, evaluator 134, and presenter 135 are not illustrated as tangible elements, but as functions implemented by processing unit 13.

Obtainer 131 obtains specific sleep information. The specific sleep information is sleep information relating to the sleep of specific user 100. As described above, in the present embodiment, terminal device 20 communicates with sensor 30, thereby obtaining the specific sleep information from sensor 30. It is thus possible for obtainer 131 to obtain the specific sleep information from terminal device 20. Obtainer 131 causes storage 12 to store the specific sleep information obtained from terminal device 20. Sleep log 121 stored in storage 12 is a log of specific sleep information.

Evaluation criterion determiner 132 determines individual evaluation criterion 122 on the basis of sleep log 121 including the log of the specific sleep information relating to the sleep of specific user 100.

Individual evaluation criterion 122 includes an evaluation criterion for each of the sleep-related items. An evaluation condition is set on an evaluation criterion basis. Individual evaluation criterion 122 includes an evaluation condition on an evaluation criterion basis. The sleep-related items of individual evaluation criterion 122 are the same as the sleep-related items of general evaluation criterion 123. Thus, the sleep-related items of individual evaluation criterion 122 correspond to sleep duration, sleep efficiency, sleep latency (time taken to fall asleep), the number of mid-sleep awakenings, the state immediately before awakening, and the ratio of deep sleep. As with general evaluation criterion 123, individual evaluation criterion 122 includes an evaluation condition for sleep duration, an evaluation condition for sleep efficiency, an evaluation condition for sleep latency, an evaluation condition for the number of mid-sleep awakenings, an evaluation condition for the state immediately before awakening, and an evaluation condition for the ratio of deep sleep. The result of a sleep evaluation that uses individual evaluation criterion 122 is a total of: an evaluation value based on the evaluation condition for sleep duration (sleep duration evaluation value) Vs1; an evaluation value based on the evaluation condition for sleep efficiency (sleep efficiency evaluation value) Vs2; an evaluation value based on the evaluation condition for sleep latency (sleep latency evaluation value) Vs3; an evaluation value based on the evaluation condition for the number of mid-sleep awakenings (mid-sleep awakening evaluation value) Vs4; an evaluation value based on the evaluation condition for the state immediately before awakening (state immediately before awakening evaluation value) Vs5; and an evaluation value based on the evaluation condition for the ratio of deep sleep (deep sleep ratio evaluation value) Vs6. Thus, supposing that the result of a sleep evaluation that uses individual evaluation criterion 122 is Vs, it is written as Vs=ΣVsn, where n is equal to 1 through 6.

In the present embodiment, evaluation criterion determiner 132 determines individual evaluation criterion 122 so that statical information relating to the evaluation values obtained from sleep log 121 satisfies a predetermined condition. Evaluation criterion determiner 132 determines, for individual evaluation criterion 122, an evaluation condition for sleep duration, an evaluation condition for sleep efficiency, an evaluation condition for sleep latency, an evaluation condition for the number of mid-sleep awakenings, an evaluation condition for the state immediately before awakening, and an evaluation condition for the ratio of deep sleep. Evaluation criterion determiner 132 uses general evaluation criterion 123 in determining individual evaluation criterion 122. Using general evaluation criterion 123, evaluation criterion determiner 132 determines an evaluation value of the sleep information (specific sleep information) included in sleep log 121 on an evaluation condition basis. Evaluation criterion determiner 132 determines statical information of evaluation values on an evaluation condition basis. The statical information can include the mean value, the variance value, and the standard deviation of the evaluation values. The statical information is determined as satisfying the predetermined condition when each of the mean value, the variance value, and the standard deviation included in the statical information is within a predetermined range. Evaluation criterion determiner 132 adjusts the evaluation conditions so that the statical information satisfies the predetermined condition.

A method of determining an evaluation condition for the number of mid-sleep awakenings is shown as an example. The evaluation condition of general evaluation criterion 123 is as shown in Table 1 below.

TABLE 1

| The number of mid-sleep | |
|---|---|
| awakenings | Evaluation value |
| 0 | 10 |
| 1 | 7 |
| 2 | 5 |
| 3 | 0 |
| 4 or more | 0 |

Using the evaluation condition for the number of mid-sleep awakenings of general evaluation criterion 123, evaluation criterion determiner 132 determines evaluation values of the sleep information (specific sleep information) included in sleep log 121. Evaluation criterion determiner 132 further determines statical information of the evaluation values. Here, the statical information includes the mean value and the standard deviation of the evaluation values. Evaluation criterion determiner 132 searches for the evaluation condition for the number of mid-sleep awakenings with which the mean value is included in a range between 7.4 and 7.7 and the standard deviation is included in a range between 2.8 and 3.2. More specifically, evaluation criterion determiner 132 generates evaluation conditions in which evaluation values are changed for the number of mid-sleep awakenings, determines statical information of the evaluation values under the generated evaluation conditions, and determines whether the determined statical information satisfies a predetermined condition. Evaluation criterion determiner 132 repeats the process of changing evaluation values for the number of mid-sleep awakenings until when an evaluation condition is obtained under which statical information satisfies the predetermined condition. Through this, evaluation criterion determiner 132 determines the evaluation condition for the number of mid-sleep awakenings to be used as individual evaluation criterion 122. When the statical information obtained under the evaluation condition shown in Table 2 below satisfies the predetermined condition, evaluation criterion determiner 132 employs the evaluation condition shown in Table 2 below.

TABLE 2

| The number of mid-sleep | |
|---|---|
| awakenings | Evaluation value |
| 0 | 10 |
| 1 | 8 |
| 2 | 6 |
| 3 | 4 |
| 4 or more | 0 |

Evaluation criterion determiner 132 determines individual evaluation criterion 122 when a certain amount of specific sleep information has been accumulated. In the present embodiment, evaluation criterion determiner 132 determines individual evaluation criterion 122 when the number of days of specific sleep information included in sleep log 121 reaches a predetermined number of days. The predetermined number of days is simply required to be the number of days that is enough to obtain a sufficient amount of specific sleep information for determining individual evaluation criterion 122. The predetermined number of days may be, for example, 30 days or more.

Individual evaluation criterion 122 determined by evaluation criterion determiner 132 is to be stored in storage 12.

Weight determiner 133 determines weight information 124 for each of general evaluation criterion 123 and individual evaluation criterion 122. In the present embodiment, weight information 124 is information used to assign weights to evaluation values. General evaluation criterion 123 and individual evaluation criterion 122 each include evaluation criteria for the respective sleep-related items. Weight information 124 includes weights for the respective evaluation criteria. Each of the weights is a coefficient (weight coefficient) to be multiplied to an evaluation value. Here, supposing that a weight coefficient for individual evaluation criterion 122 is ws and a weight coefficient for general evaluation criterion 123 is wg for the same evaluation value, ws is a value between 0 and 1, inclusive, and ws+wg=1. To be more specific, weight information 124 includes weight coefficients ws1, ws2, ws3, ws4, ws5, and ws6 of individual evaluation criterion 122 corresponding to sleep duration evaluation value Vs1, sleep efficiency evaluation value Vs2, sleep latency evaluation value Vs3, mid-sleep awakening evaluation value Vs4, state immediately before awakening evaluation value Vs5, and deep sleep ratio evaluation value Vs6, respectively. Weight information 124 includes weight coefficients wg1, wg2, wg3, wg4, wg5, and wg6 of general evaluation criterion 123 corresponding to sleep duration evaluation value Vg1, sleep efficiency evaluation value Vg2, sleep latency evaluation value Vg3, mid-sleep awakening evaluation value Vg4, state immediately before awakening evaluation value Vg5, and deep sleep ratio evaluation value Vg6, respectively. Here, each value of wsn is between 0 and 1, inclusive, where n is equal to 1 through 6. Thus, wsn+wgn=1, where n is equal to 1 through 6.

Weight determiner 133 determines weight information 124 (ws 1 through ws6 and wg1 through wg6), on the basis of the result of comparing a first representative value and a second representative value.

The first representative value is a representative value of the results of evaluating the sleep of specific user 100 using general evaluation criterion 123. The representative value can be selected, as appropriate, from the maximum value, the minimum value, the mean value, the mode value, etc. In the present embodiment, the representative value is the mean value. For example, using general evaluation criterion 123, weight determiner 133 determines a plurality of results (evaluation values) of evaluating the sleep of specific user 100 from items of specific sleep information, and uses the representative value of such plurality of results (evaluation values) as the first representative value. When evaluation values are sleep efficiency evaluation values Vg2, for example, the mean value of sleep efficiency evaluation values Vg2 serves as the first representative value. Weight determiner 133 determines the first representative value when the number of items of specific sleep information included in sleep log 121 reaches a predetermined value. Stated differently, using general evaluation criterion 123, weight determiner 133 evaluates the items of specific sleep information accumulated during a predetermined period (during a predetermined number of days) to obtain the representative value (first representative value) of a plurality of evaluation results (evaluation values) of specific user 100.

The second representative value is a representative value of the results of evaluating the sleep of a plurality of users 200 using general evaluation criterion 123. The representative value can be selected as appropriate from the maximum value, the minimum value, the mean value, the mode value, etc. In the present embodiment, the representative value is the mean value. For example, using general evaluation criterion 123, weight determiner 133 determines a plurality of results (evaluation values) of evaluating the sleep of a plurality of users 200 from the sleep information of a plurality of users 200, and uses the representative value of such plurality of results (evaluation values) as the second representative value. When evaluation values are sleep efficiency evaluation values Vg2, for example, the mean value of sleep efficiency evaluation values Vg2 serves as the second representative value. The sleep information relating to a plurality of users 200 may be items of sleep information accumulated during the same predetermined period as that of the specific sleep information.

As described above, weight determiner 133 determines weight information 124 (ws 1 through ws6 and wg1 through wg6) on the basis of: the result of evaluating the sleep of specific user 100 using general evaluation criterion 123 that is based on the items of specific sleep information accumulated during a predetermined or greater number of days (the first representative value); and the result of evaluating the sleep of a plurality of users 200 (ordinary users) using general evaluation criterion 123 (the second representative value).

When the first representative value and the second representative value are equal, each of the weights (ws1 through ws6) of individual evaluation criterion 122 is set to a specified value. When no significant difference is present between the first representative value and the second representative value, these values can be regarded as equal. The specified value is, for example, 0.7. When the first representative value is better than the second representative value, each of the weights (ws1 through ws6) of individual evaluation criterion 122 is set to a value smaller than the specified value. The value smaller than the specified value is, for example, 0.3. When the first representative value is worse than the second representative value, each of the weights (ws1 through ws6) of individual evaluation criterion 122 is set to a value greater than the specified value. The value greater than the specified value is, for example, 10.

Weight determiner 133 uses weight information 124 (ws1 through ws6 and wg1 through wg6) determined in the above manner as goal weight information 124. In so doing, weight determiner 133 reflects goal weight information 124 on evaluations over transition time from current weight information 124. The transition time is, for example, on the order of one week, during which weight information 124 is gradually changed from the current values to the goal values. For example, the weights may be changed from the current values to the goal values either linearly or curvaceously. This enables specific user 100 not to be aware of changes in evaluation values attributable to changes in weight information 124. As described above, after determining weight information (goal weight information) 124, weight determiner 133 in the present embodiment reflects goal weight information 124 on evaluations over the transition time.

Weight determiner 133 updates weight information 124 when a predetermined condition is satisfied. The predetermined condition is, for example, that the number of days of items of specific sleep information included in sleep log 121 has increased as many as a specified number of days. Alternatively, the predetermined condition may be the timing of receiving an instruction for updating weight information 124. An instruction for updating weight information 124 is provided by terminal device 20.

Evaluator 134 performs an evaluation process. The evaluation process is a process of evaluating the sleep of specific user 100 from the specific sleep information, using both individual evaluation criterion 122 and general evaluation criterion 123. Evaluator 134 uses weight information 124 to perform the evaluation process. Using individual evaluation criterion 122, evaluator 134 determines Vs1 through Vs6 from the specific sleep information. Using general evaluation criterion 123, evaluator 134 determines Vg1 through Vg6 from the specific sleep information. Evaluator 134 assigns weights to Vs1 through Vs6 and Vg1 through Vg6 using weight information 124 and evaluates the sleep of specific user 100. Supposing that the result of evaluating the sleep of specific user 100 is Vt, it is written as Vt=ΣVsn×wsn+Vgn×wgn. When Vs1 is 27 points, Vs2 is 20 points, Vs3 is 7 points, Vs4 is 8 points, Vs5 is 6 points, and Vs6 is 5 points, for example, result Vs of evaluating the sleep of specific user 100 using individual evaluation criterion 122 is 73 points. When Vg1 is 20 points, Vg2 is 17 points, Vg3 is 7 points, Vg4 is 8 points, Vg5 is 6 points, and Vg6 is 7 points, for example, result Vg of evaluating the sleep of specific user 100 using general evaluation criterion 123 is 65 points. When ws1 through ws6 are all 0.7, result Vt of evaluating the sleep of specific user 100 using both individual evaluation criterion 122 and general evaluation criterion 123 is 70.6.

In the evaluation process, evaluator 134 determines result Vt of evaluating the sleep of specific user 100 using both individual evaluation criterion 122 and general evaluation criterion 123. Evaluator 134 further determines result Vs of evaluating the sleep of specific user 100 using individual evaluation criterion 122 and result Vg of evaluating the sleep of specific user 100 using general evaluation criterion 123. The result of the evaluation process thus includes a first evaluation result indicating Vs and Vg and a second evaluation result indicating Vt.

After weight information 124 is changed, evaluator 134 performs an evaluation process again using the changed weight information 124. Through this, evaluator 134 reflects the change in weight information 124 also on the past evaluations. For example, evaluator 134 calculates Vt using the changed weight information 124 on the basis of the specific sleep information included in sleep log 121. As a result, after the change of weight information 124, Vt corresponding to the specific sleep information of the same date in the past also changes. With this, it is possible for evaluator 134 to perform a consistent evaluation process.

Before evaluation criterion determiner 132 determines individual evaluation criterion 122, evaluator 134 uses general evaluation criterion 123 to evaluate the sleep of specific user 100. This is based on the consideration that evaluation criterion determiner 132 determines individual evaluation criterion 122 when a certain amount of specific sleep information has been accumulated. This enables evaluator 134 to present the evaluation result to specific user 100 even before a certain amount of specific sleep information has been accumulated.

Presenter 135 presents the result of the evaluation process performed by evaluator 134. As described above, the result of the evaluation process includes: the first evaluation result indicating results Vs and Vg of evaluating the sleep of specific user 100 using one of individual evaluation criterion 122 and general evaluation criterion 123; and the second evaluation result indicating result Vt of evaluating the sleep of specific user 100 using both individual evaluation criterion 122 and general evaluation criterion 123. Presenter 135 is also capable of presenting a comparison of the first evaluation result and the second evaluation result. This enables an easy grasp of a sleep evaluation. Presenter 135 serves as an advice generator that generates an advice on sleep to specific user 100. For example, the advice generator may determine the sleep type of specific user 100 from sleep log 121 to generate an advice appropriate to the sleep type. Examples of the sleep type include the types of: having difficulty in falling asleep; having difficulty in waking up; waking up during sleep; waking up early in the morning; and having light sleep. An advice on sleep is generated by a variety of known methods, and thus will not be described in detail.

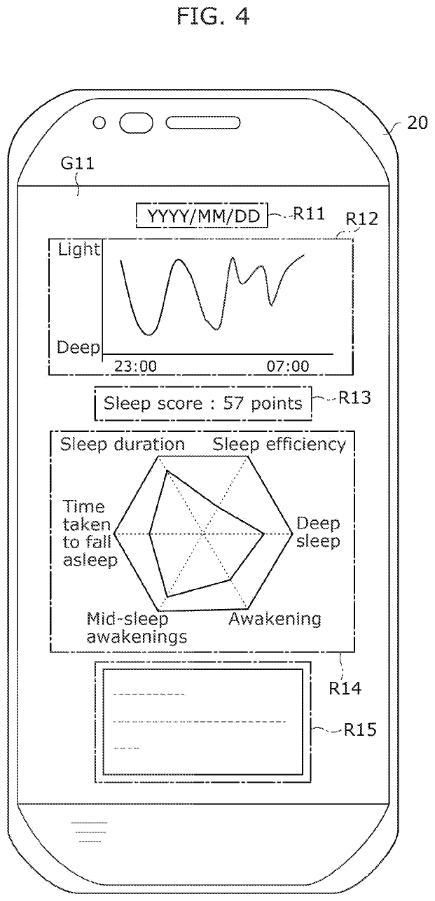

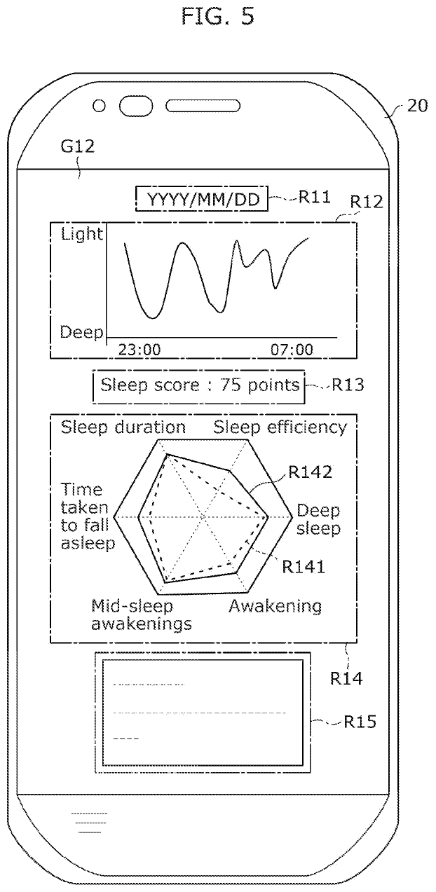

FIG. 4 and FIG. 5 show exemplary screens (result display screens) displayed by presenter 135. In FIG. 4 and FIG. 5, the result display screens are displayed on the image display device of terminal device 20. Result display screen G11 shown in FIG. 4 is a screen that presents the result of an evaluation process performed before evaluation criterion determiner 132 determines individual evaluation criterion 122. Result display screen G11 includes first region R11, second region R12, third region R13, fourth region R14, and fifth region R15. First region R11 shows the date of specific sleep information that has undergone the evaluation process performed by evaluator 134. Second region R12 displays a graph representing an outline of the specific sleep information. In FIG. 4, second region R12 shows, in a graph form, temporal changes in the sleep depth and the sleep duration. Third region R13 is a region that presents the result of the evaluation process. In FIG. 4, third region R13 displays, as a sleep score, result Vg (firs evaluation result) of evaluating the sleep of specific user 100 using general evaluation creation 123. Fourth region R14 displays, as a radar chart, the details of the sleep score (here, sleep duration evaluation value Vg1, sleep efficiency evaluation value Vg2, sleep latency evaluation value Vg3, mid-sleep awaking evaluation value Vg4, state immediately before awakening evaluation value Vg5, and deep sleep ratio evaluation value Vg6). Fifth region R15 is a region for displaying a text relating to an advice on sleep to specific user 100. The advice is generated by presenter 135.

Result display screen G12 shown in FIG. 5 is a screen that presents the result of an evaluation process performed after evaluation criterion determiner 132 determines individual evaluation criterion 122. As with result display screen G11, result display screen G12 includes first region R11, second region R12, third region R13, fourth region R14, and fifth region R15. In FIG. 5, third region R13 displays, as a sleep score, result Vt of evaluating the sleep of specific user 100 using both individual evaluation criterion 122 and general evaluation criterion 123. Fourth region R14 displays, as a radar chart, the details of the sleep score. Fourth region R14 includes first radar chart 141 and second radar chart 142. First radar chart 141 corresponds to result Vg of evaluating the sleep of specific user 100 using general evaluation criterion 123. Second radar chart 142 corresponds to result Vt of evaluating the sleep of specific user 100 using both individual evaluation criterion 122 and general evaluation criterion 123. Stated differently, fourth region R14 presents a comparison of the first evaluation result and the second evaluation result.

(1-3) Operation

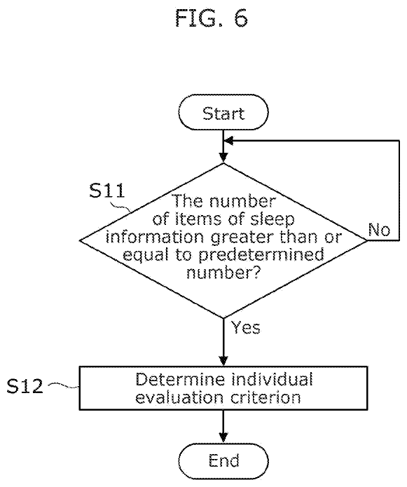

With reference to FIG. 6 through FIG. 20, the following briefly describes an exemplary operation performed by sleep evaluation system 1.

FIG. 6 is a flowchart of an operation performed by sleep evaluation system 1 of determining individual evaluation criterion 122. Evaluation criterion determiner 132 determines whether the number of items of specific sleep information included in sleep log 121 has reached a predetermined number (S11). When the number of the items of specific sleep information included in sleep log 121 has reached the predetermined number (S11: Yes), evaluation criterion determiner 132 determines individual evaluation criterion 122 (S12).

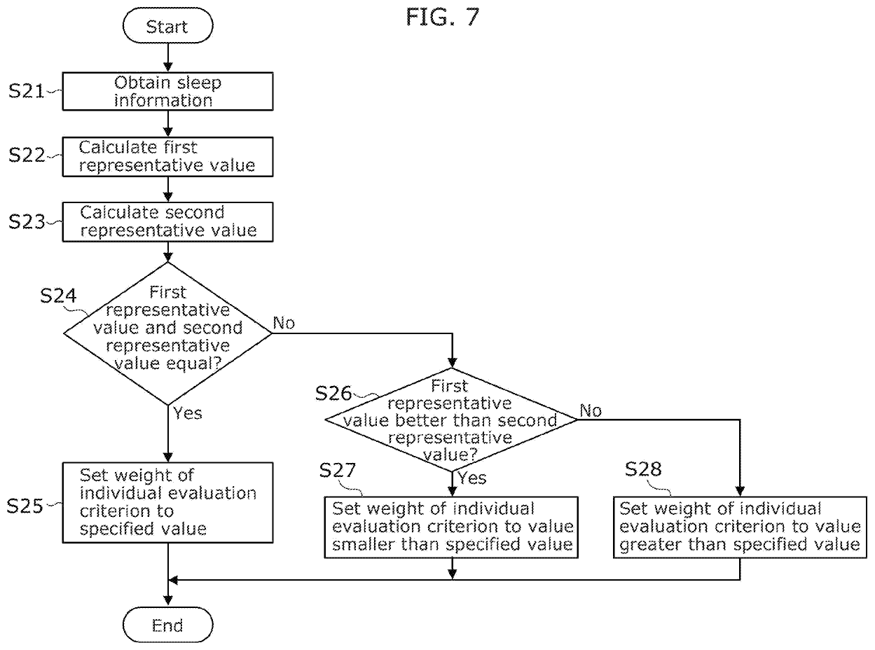

After individual evaluation criterion 122 is determined, it is possible to determine weight information 124. FIG. 7 is a flowchart of an operation performed by sleep evaluation system 1 of determining weight information. Weight determiner 133 obtains specific sleep information from sleep log 121 (S21). Using general evaluation criterion 123, weight determiner 133 determines, from the obtained specific sleep information, the first representative value of the results of evaluating the sleep of specific user 100 (S22). Using general evaluation criterion 123, weight determiner 133 determines, from the sleep information of a plurality of users 200, the second representative value of the results of evaluating the sleep of a plurality of users 200 (S23). Weight determiner 133 compares the first representative value and the second representative value (S24, S26). When the first representative value and the second representative value are equal (S24: YES), weight determiner 133 sets each of the weights of individual evaluation criterion 122 (ws1 through ws6) to the specified value (S25). When the first representative value is better than the second representative value (S24: NO, S26: YES), weight determiner 133 sets each of the weights of individual evaluation criterion 122 (ws1 through ws6) to a value smaller than the specified value (S27). When the first representative value is worse than the second representative value (S24: NO, S26: NO), weight determiner 133 sets each of the weights of individual evaluation criterion 122 (ws1 through ws6) to a value greater than the specified value (S28).

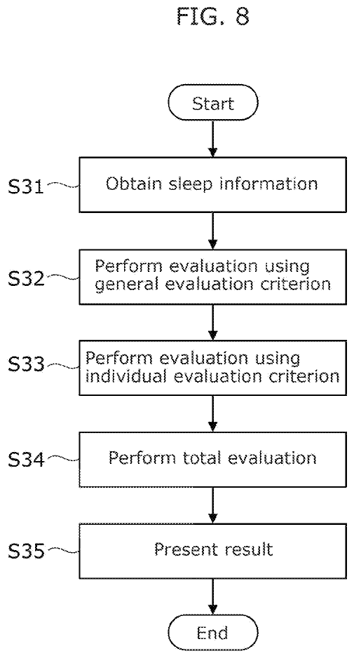

FIG. 8 is a flowchart of an operation performed by sleep evaluation system 1 of evaluating the sleep of specific user 100. Evaluator 134 obtains the specific sleep information from sleep log 121 (S31). Evaluator 134 performs, for the obtained specific sleep information, an evaluation of the sleep of specific user 100 using general evaluation criterion 123 (S32) and an evaluation of the sleep of specific user 100 using individual evaluation criterion 122 (S33). Evaluator 134 obtains result Vt of evaluating the sleep of specific user 100 using both individual evaluation criterion 122 and general evaluation criterion 123 (S34) on the basis of: the results of the evaluation that uses general evaluation criterion 123 (e.g., Vg1 through Vg6); the results of the evaluation that uses individual evaluation criterion 122 (e.g., Vs1 through Vs6); and weight information (ws1 through ws6). As described above, evaluator 134 performs an evaluation process of evaluating, from the specific sleep information, the sleep of specific user 100 using individual evaluation criterion 122, general evaluation criterion 123, and weight information 124 to obtain result Vt of evaluating the sleep of specific user 100. Presenter 135 presents the result of the evaluation performed by evaluator 134 (i.e., the result of the evaluation process) (S35).

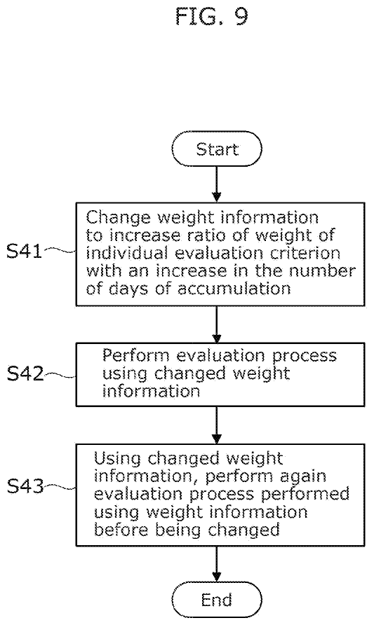

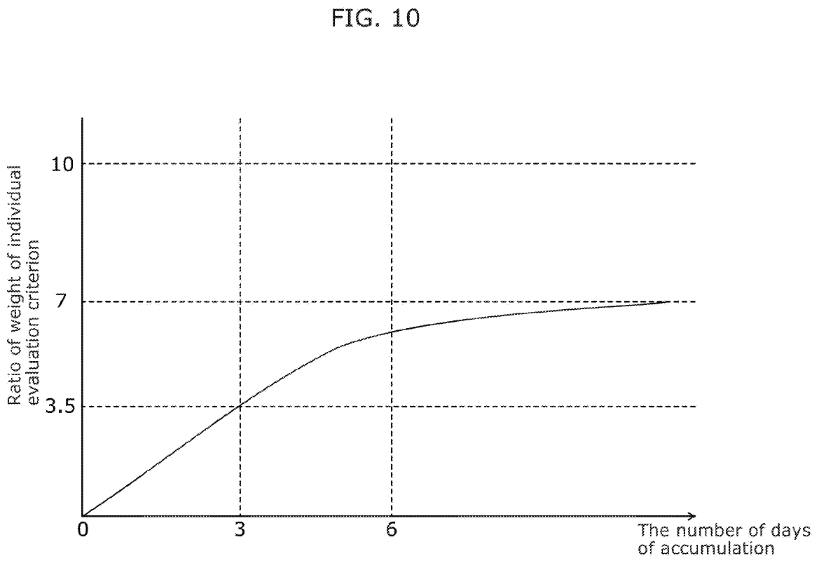

FIG. 9 is a flowchart of an operation performed by sleep evaluation system 1 of changing weight information 124 on the basis of the number of day of accumulation and performing an evaluation using the changed weight information 124. FIG. 10 is a diagram showing an exemplary graph representing the ratio of the weight of individual evaluation criterion 122 to the number of days of accumulation.

Weight determiner 133 changes weight information 124 to increase the ratio of the weight of individual evaluation criterion 122 to the total of the weight of individual evaluation criterion 122 and the weight of general evaluation criterion 123 with an increase in the number of days of accumulation for which items of specific sleep information has been accumulated (S41). Stated differently, weight determiner 133 changes weight information 124 to decrease the ratio of the weight of general evaluation criterion 123 to the total of the weight of individual evaluation criterion 122 and the weight of general evaluation criterion 123 with an increase in the number of days of accumulation. As shown in FIG. 10, for example, weight determiner 133 linearly increases the ratio of the weight of individual evaluation criterion 122. In so doing, weight determiner 133 determines that: the ratio of the weight of individual evaluation criterion 122 is 0, i.e., the ratio of the weight of general evaluation criterion 123 is 10, when the number of days of accumulation is 0 days; and the ratio of the weight of individual evaluation criterion 122 is 3.5, i.e., the ratio of the weight of general evaluation criterion 123 is 6.5, when the number of days of accumulation is 3 days. More specifically, for example, when the number of days of accumulation is 0 days, weight determiner 133 determines that wsn=0 and wgn=10, and when the number of days of accumulation is 3 days, weight determiner 133 determines that wsn=3.5 and wgn=6.5. As described above, weight determiner 133 changes weight information 124 on the basis of the number of days of accumulation.

Evaluator 134 performs an evaluation process using the changed weight information 124 (S42).

Also, using the changed weight information 124, evaluator 134 performs again the evaluation process that has been performed using weight information 124 before being changed (S43).

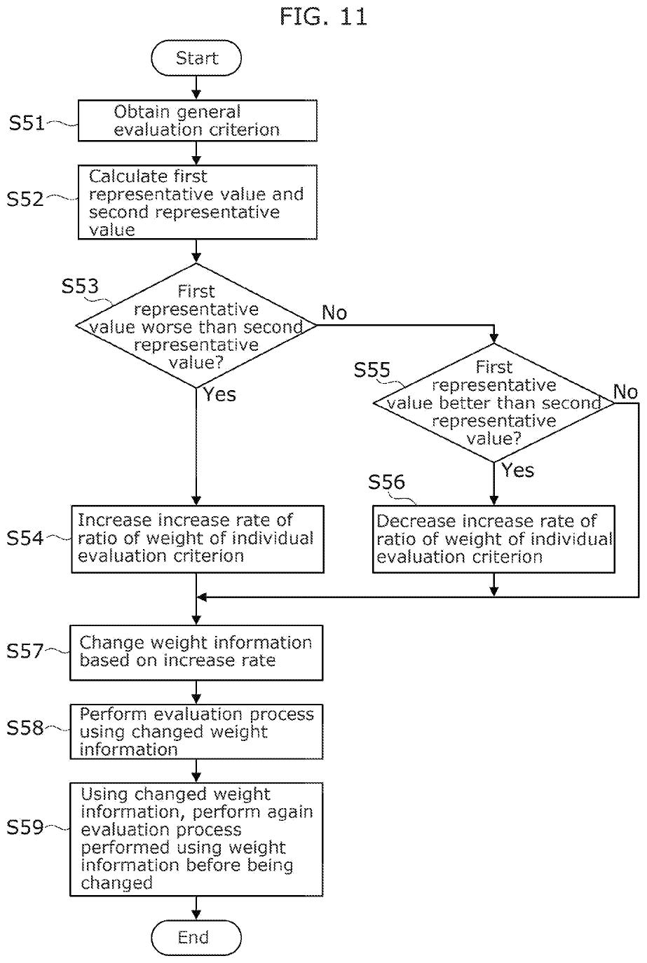

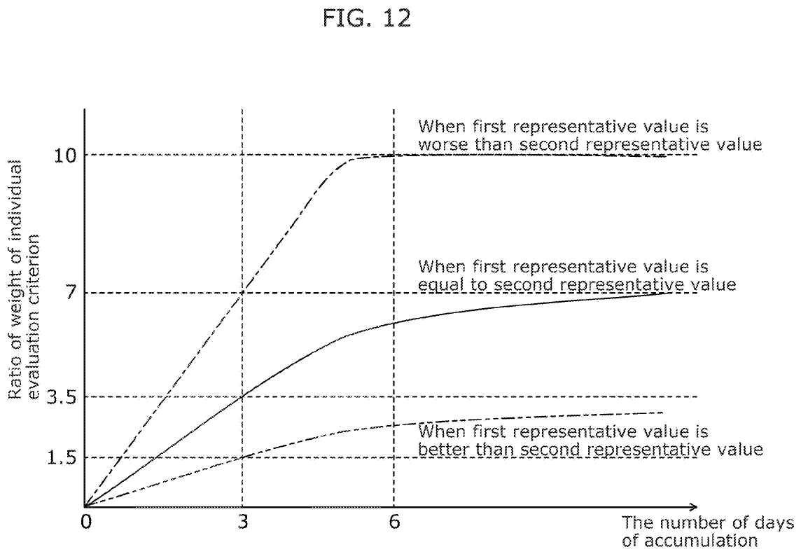

FIG. 11 is a flowchart of an operation performed by sleep evaluation system 1 of changing weight information 124 on the basis of the number of days of accumulation and performing an evaluation using the changed weight information 124. FIG. 12 is a diagram showing an exemplary graph representing the ratio of the weight of individual evaluation criterion 122 to the number of days of accumulation.

Weight determiner 133 obtains general evaluation criterion 123 (S51) and calculates the first representative value and the second representative value, using the obtained general evaluation criterion 123 (S52). In the present embodiment, as described above, the first representative value is the mean value of the results of evaluating the sleep of specific user 100 using general evaluation criterion 123 and the second representative value is the mean value of the results of evaluating the sleep of a plurality of users 200 using general evaluation criterion 123. When the number of mid-sleep awakenings of specific user 100 for 10 days and the number of mid-sleep awakenings of a plurality of users 200 for the same 10 days are used, for example, the first representative value is the mean value of the evaluation values of the number of mid-sleep awakenings of specific user 100 for the 10 days calculated using general evaluation criterion 123 and the second representative value is the mean value of the evaluation values of the number of mid-sleep awakenings of a plurality of users 200 for the same 10 days calculated using general evaluation criterion 123.

Weight determiner 133 compares the first representative value and the second representative value to determine whether the first representative value is worse than the second representative value (S53). When the first representative value is smaller than the second representative value, for example, weight determiner 133 determines that the first representative value is worse than the second representative value. Meanwhile, when the first representative value is greater than or equal to the second representative value, weight determiner 133 determines that the first representative value is not worse than the second representative value.

When the first representative value is worse than the second representative value (S53: YES), weight determiner 133 increases an increase rate of the ratio of the weight of individual evaluation criterion 122 to an increase in the number of days of accumulation (S54). As shown in FIG. 12, when the first representative value is worse than the second representative value, for example, weight determiner 133 more increases the increase rate (the slope of the graph) than when the first representative value is equal to the second representative value.

When the first representative value is not worse than the second representative value (S53: NO), weight determiner 133 determines whether the first representative value is better than the second representative value (S55). When the first representative value is greater than the second representative value, for example, weight determiner 133 determines that the first representative value is better than the second representative value. Meanwhile, when the first representative value is equal to the second representative value, weight determiner 133 determines that the first representative value is not better than the second representative value.

When the first representative value is better than the second representative value (S55: YES), weight determiner 133 decreases the increase rate of the ratio of the weight of individual evaluation criterion 122 to an increase in the number of days of accumulation (S56). As shown in FIG. 12, when the first representative value is better than the second representative value, for example, weight determiner 133 more decreases the increase rate (the slope of the graph) than when the first representative value is equal to the second representative value.

As described above, weight determiner 133 changes the increase rate on the basis of the result of comparing the first representative value of the results of evaluating the sleep of specific user 100 using general evaluation criterion 123 and the second representative value of the results of evaluating the sleep of a plurality of users 200 using general evaluation criterion 123.

When the first representative value is not better than the second representative value (S55: NO), i.e., when the first representative value is equal to the second representative value, weight determiner 133 does not change the increase rate of the ratio of the weight of individual evaluation criterion 122 to an increase in the number of days of accumulation.

Weight determiner 133 changes weight information 124 on the basis of the increase rate of the ratio of the weight of individual evaluation criterion 122 to an increase in the number of days of accumulation (S57). When having changed the increase rate, weight determiner 133 changes weight information 124 on the basis of the changed increase rate. When having changed the increase rate as shown in the dash-dot-dash line shown in FIG. 12, for example, weight determiner 133 changes the weight information so that wsn=7 and wgn=3 when the number of day of accumulation of is 3 days. When having changed the increase rate as shown in the dash-dot-dot-dash line shown in FIG. 12, weight determiner 133 changes the weight information so that wsn=1.5 and wgn=8.5 when the number of days of accumulation of is 3 days.

Evaluator 134 uses the changed weight information 124 to perform an evaluation process (S58).

Also, using the changed weight information 124, evaluator 134 performs again the evaluation process that has been performed using weight information 124 before being changed (S59).

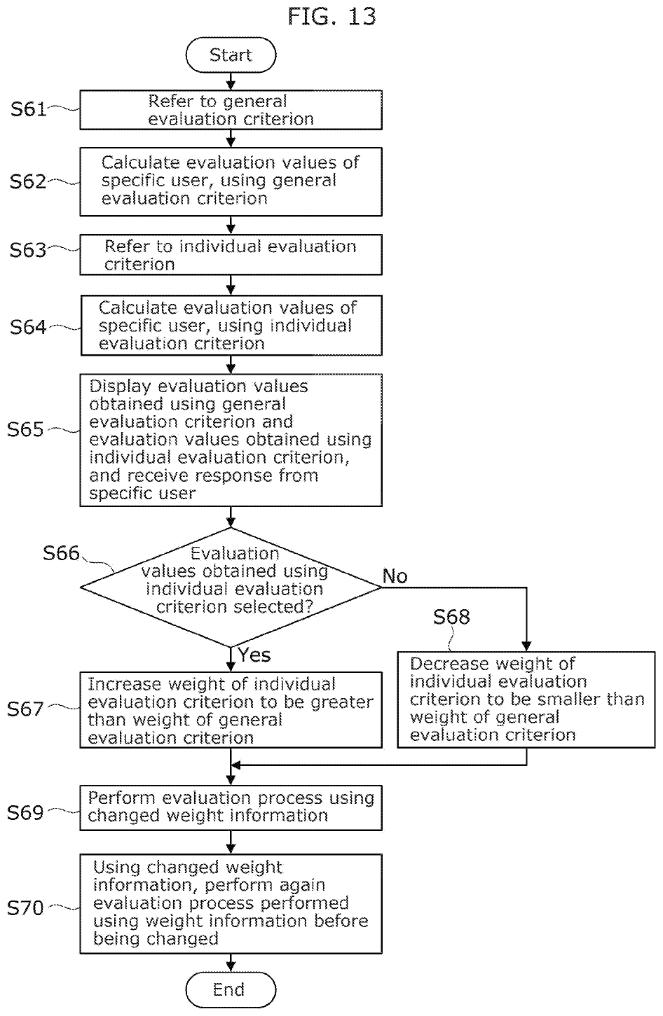

FIG. 13 is a flowchart of an operation performed by sleep evaluation system 1 of changing weight information 124 on the basis of a response from specific user 100 and performing an evaluation using the changed weight information 124.

Evaluator 134 refers to general evaluation criterion 123 (S61) and calculates evaluation values of specific user 100 using general evaluation criterion 123 (S62). For example, using general evaluation criterion 123, evaluator 134 calculates evaluation values of the respective items of specific user 100 (sleep duration, sleep efficiency, sleep latency (time taken to fall asleep), the number of mid-sleep awakenings, the state immediately before awakening, and the ratio of deep sleep).

Evaluator 134 refers to individual evaluation criterion 122 (S63) and calculates evaluation values of specific user 100 using individual evaluation criterion 122 (S64). For example, using individual evaluation criterion 122, evaluator 134 calculates evaluation values of the respective items of specific user 100 (sleep duration, sleep efficiency, sleep latency (time taken to fall asleep), the number of mid-sleep awakenings, the state immediately before awakening, and the ratio of deep sleep).

Presenter 135 displays the evaluation values calculated using general evaluation criterion 123 and the evaluation values calculated using individual evaluation criterion 122 and receives a response from specific user 100 (S65). As described above, presenter 135 presents, to specific user 100, the result of evaluating the sleep of specific user 100 using individual evaluation criterion 122 and the result of evaluating the sleep of specific user 100 using general evaluation criterion 123 and receives a response from specific user 100.

Weight determiner 133 determines whether specific user 100 has selected the evaluation values calculated using individual evaluation criterion 122 (S66).

When specific user 100 has selected the evaluation values calculated using individual evaluation criterion 122 (YES in S66), weight determiner 133 increases the weight of individual evaluation criterion 122 to be greater than the weight of general evaluation criterion 123 (S67). For example, weight determiner 133 increases the weight to be wsn>wgn. As described above, when specific user 100 has selected the result of evaluating the sleep of specific user 100 using individual evaluation criterion 122, weight determiner 133 increases the weight of individual evaluation criterion 122 to be greater than the weight of general evaluation criterion 123.

When specific user 100 has not selected the evaluation values calculated using individual evaluation criterion 122 (NO in S66), i.e., when specific user 100 has selected the evaluation values calculated using general evaluation criterion 123, weight determiner 133 decreases the weight of individual evaluation criterion 122 to be smaller than the weight of general evaluation criterion 123 (S68). For example, weight determiner 133 decreases the weight to be wsn<wgn. As described above, when specific user 100 has selected the result of evaluating the sleep of specific user 100 using general evaluation criterion 123, weight determiner 133 decreases the weight of individual evaluation criterion 122 to be smaller than the weight of general evaluation criterion 123.

Evaluator 134 performs an evaluation process using the changed weight information 124 (S69).

Also, using the changed weight information 124, evaluator 134 performs again the evaluation process that has been performed using weight information 124 before being changed (S70).

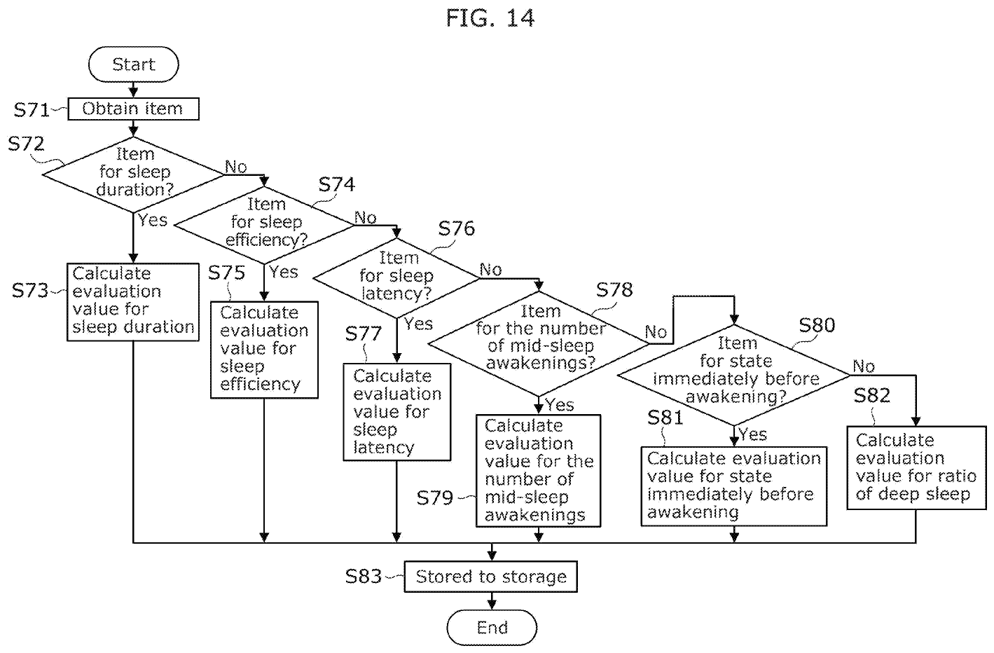

FIG. 14 is a flowchart of an operation performed by sleep evaluation system 1 of calculating evaluation values.

Evaluator 134 obtains a sleep-related item (S71). Evaluator 134 determines whether the obtained item is sleep duration (S72). When the obtained item is sleep duration (YES in S72), evaluator 134 calculates an evaluation value for sleep duration (S73).

When the obtained item is not sleep duration (NO in S72), evaluator 134 determines whether the obtained item is sleep efficiency (S74). When the obtained item is sleep efficiency (YES in S74), evaluator 134 calculates an evaluation value for sleep efficiency (S75).

When the obtained item is not sleep efficiency (NO in S74), evaluator 134 determines whether the obtained item is sleep latency (S76). When the obtained item is sleep latency (YES in S76), evaluator 134 calculates an evaluation value for sleep latency (S77).

When the obtained item is not sleep latency (NO in S76), evaluator 134 determines whether the obtained item is the number of mid-sleep awakenings (S78). When the obtained item is the number of mid-sleep awakenings (YES in S78), evaluator 134 calculates an evaluation value for the number of mid-sleep awakenings (S79).

When the obtained item is not the number of mid-sleep awakenings (NO in S78), evaluator 134 determines whether the obtained item is the state immediately before awakening (S80). When the obtained item is the state immediately before awakening (YES in S80), evaluator 134 calculates an evaluation value for the state immediately before awakening (S81).

When the obtained item is not the state immediately before awakening (NO in S80), evaluator 134 calculates an evaluation value for the ratio of deep sleep (S82).

Evaluator 134 causes storage 12 to store the calculated evaluation values (S83).

Note that the order of making determinations on the items is not limited to the foregoing order.

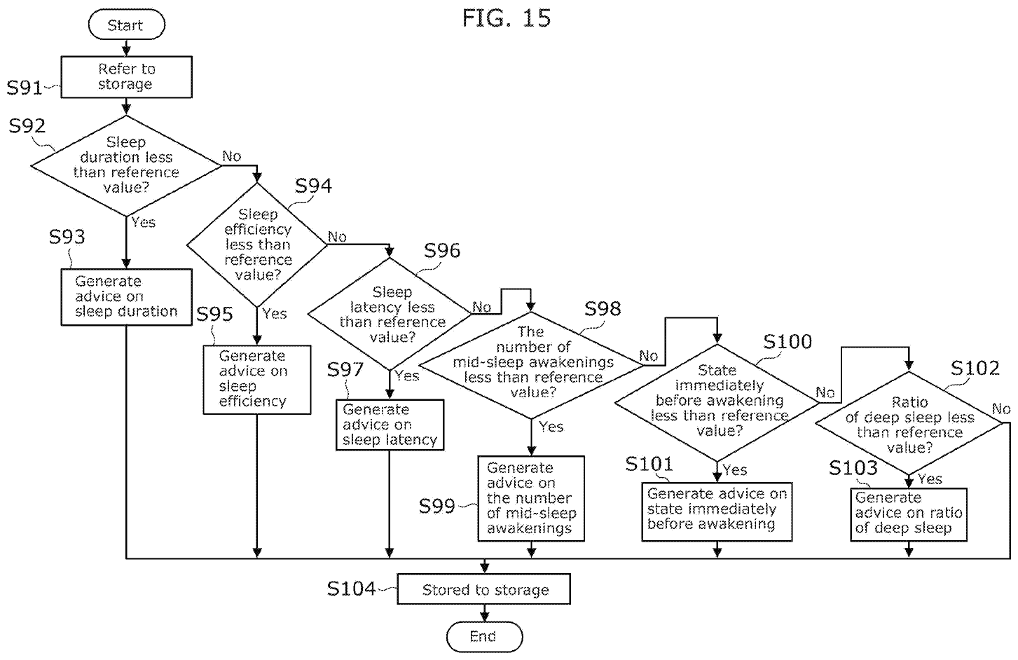

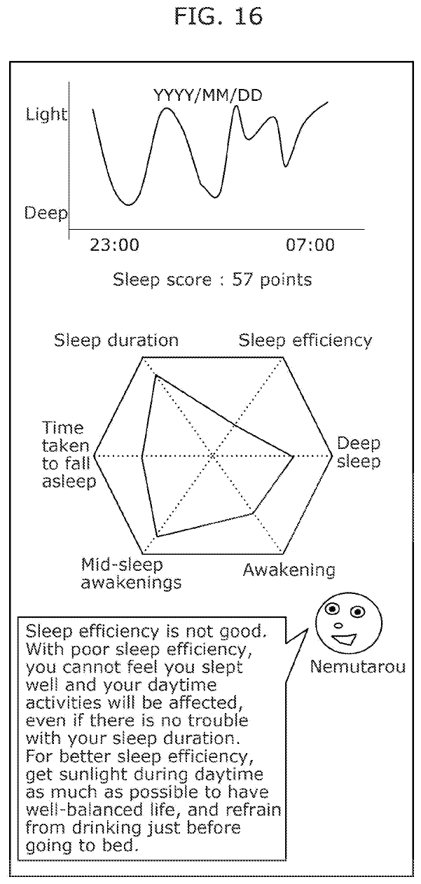

FIG. 15 is a flowchart of an operation performed by sleep evaluation system 1 of generating an advice. FIG. 16 through FIG. 18 are diagrams each for explaining an exemplary result display screen in sleep evaluation system 1.

Presenter 135 refers to storage 12 (S91) and determines whether the evaluation value for sleep duration is less than a reference value (S92). When the evaluation value for sleep duration is less than the reference value (YES in S92), presenter 135 generates an advice on sleep duration (S93). The reference value may be, for example, the 70% value of the perfect score, or the mean value of the evaluation values of specific user 100 for one week.

When the evaluation value for sleep duration is not less than the reference value (NO in S92), presenter 135 determines whether the evaluation value for sleep efficiency is less than a reference value (S94). When the evaluation value for sleep efficiency is less than the reference value (YES in S94), presenter 135 generates an advice on sleep efficiency (S95).

When the evaluation value for sleep efficiency is not less than the reference value (NO in S94), presenter 135 determines whether the evaluation value for sleep latency is less than a reference value (S96). When the evaluation value for sleep latency is less than the reference value (YES in S96), presenter 135 generates an advice on sleep latency (S97).

When the evaluation value for sleep latency is not less than the reference value (NO in S96), presenter 135 determines whether the evaluation value for the number of mid-sleep awakenings is less than a reference value (S98). When the evaluation value for the number of mid-sleep awakenings is less than the reference value (YES in S98), presenter 135 generates an advice on the number of mid-sleep awakenings (S99).

When the evaluation value for the number of mid-sleep awakenings is not less than the reference value (NO in S98), presenter 135 determines whether the evaluation value for the state immediately before awakening is less than a reference value (S100). When the evaluation value for the state immediately before awakening is less than the reference value (YES in S100), presenter 135 generates an advice on the state immediately before awakening (S101).

When the evaluation value for the state immediately before awakening is not less than the reference value (NO in S100), presenter 135 determines whether the evaluation value for the ratio of deep sleep is less than a reference value (S102). When the evaluation value for the ratio of deep sleep is less than the reference value (YES in S102), presenter 135 generates an advice on the ratio of deep sleep (S103).

When the evaluation value for the ratio of deep sleep is not less than the reference value (NO in S102), presenter 135 generates no advice.

Presenter 135 causes storage 12 to store the generated advices (S104).

Note that the order of generating the advices is not limited to the foregoing order. The advices may thus be generated starting from an item with a lower evaluation value.

As shown in FIG. 16, when the evaluation value for sleep efficiency is small compared to the evaluation values for the other items, for example, presenter 135 generates and presents an advice on sleep efficiency.

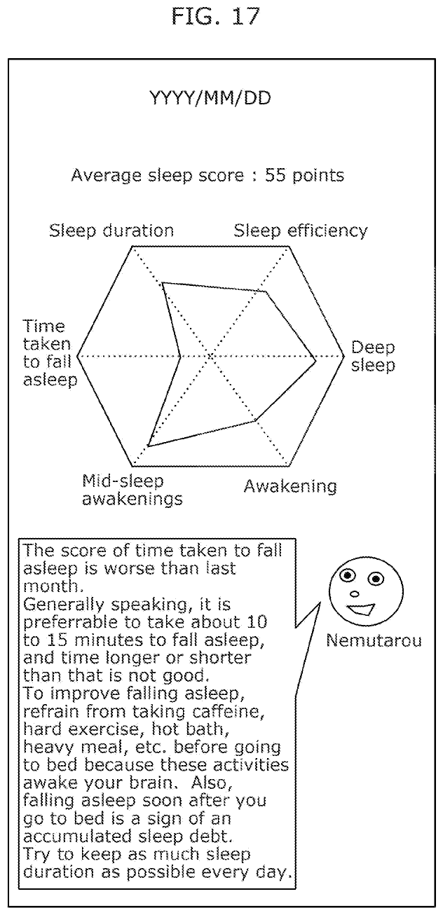

As shown in FIG. 17, when the evaluation value for time taken to fall asleep (sleep latency) is small compared to the evaluation values for the other items, for example, presenter 135 generates and presents an advice on time taken to fall asleep (sleep latency).

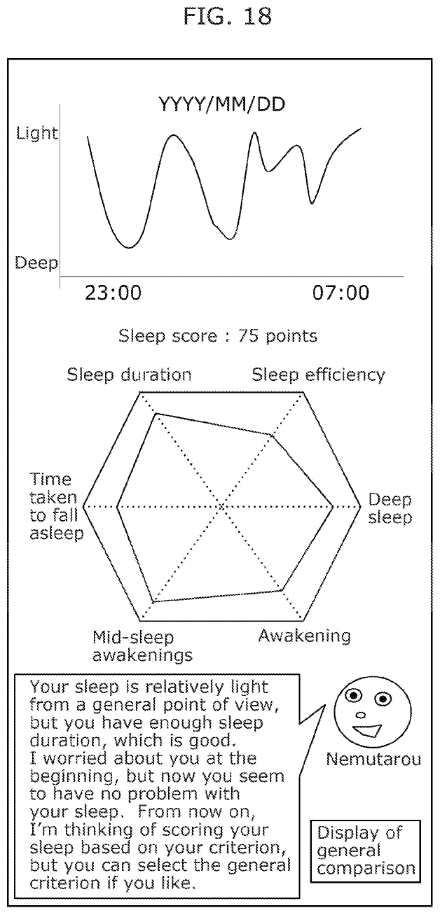

Also, as shown in FIG. 18, for example, presenter 135 generates and presents an advice on the basis of the result of comparing the evaluation values of specific user 100 and the evaluation values of a plurality of users 200.

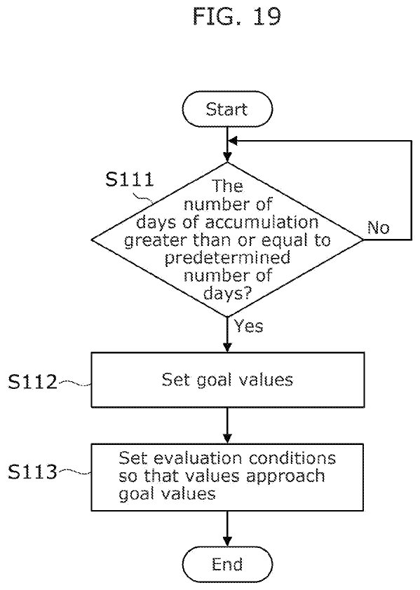

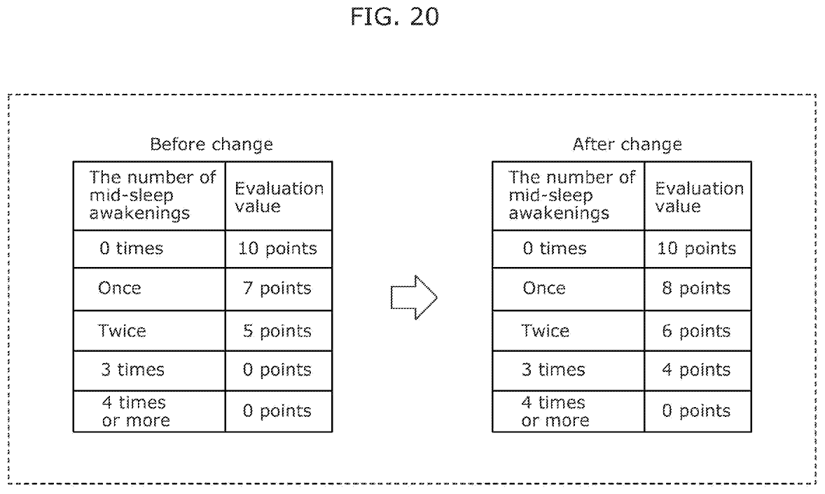

FIG. 19 is a flowchart of an operation performed by sleep evaluation system 1 of changing individual evaluation criterion 122. FIG. 20 is a diagram for explaining an example of individual evaluation criterion 122 before and after being changed.

Evaluation criterion determiner 132 determines whether the number of days of accumulation is greater than or equal to a predetermined number of days (S111). The predetermined number of days is, for example, 35 days.

When the number of days of accumulation is not greater than or equal to the predetermined number of days (NO in S111), evaluation criterion determiner 132 determines again whether the number of days of accumulation is greater than or equal to the predetermined number of days (S111). When the number of days of accumulation is greater than or equal to the predetermined number of days (YES in S111), evaluation criterion determiner 132 sets goal values (S112). The goal values can include, for example, the mean value, the variance value, etc. of the evaluation values for the predetermined number of days. More specifically, when the item is the number of mid-sleep awakenings, for example, the goal value can include, for example, the mean value, the variance value, etc. of the evaluation values of the number of mid-sleep awakenings during the predetermined number of days.

Evaluation criterion determiner 132 sets evaluation conditions so that evaluation values approach the set goal values (S113). To change the evaluation condition for the number of mid-sleep awakenings, for example, evaluation criterion determiner 132 searches for an evaluation condition for the number of mid-sleep awakenings that is included in the mean value range between 7.4 and 7.7 and the standard deviation range between 2.8 and 3.2. As shown in FIG. 20, for example, when the goal value has been achieved as a result of setting 10 points for 0 times, 8 points for once, 6 points for twice, 4 points for 3 times, and 0 points for 4 times or more, evaluation criterion determiner 132 changes the evaluation condition for the number of mid-sleep awakenings to stipulate that 10 points for 0 times, 8 points for once, 6 points for twice, 4 points for 3 times, and 0 points for 4 times or more.

(1-4) Summary

As described above, sleep evaluation system 1 includes evaluation criterion determiner 132 and evaluator 134. Evaluation criterion determiner 132 determines an individual evaluation criterion on the basis of sleep log 121 that includes a log of specific sleep information relating to the sleep of specific user 100. Evaluator 134 performs an evaluation process of evaluating the sleep of specific user 100 from the specific sleep information, using both the individual evaluation criterion and a general evaluation criterion that is determined on the basis of sleep information relating to the sleep of a plurality of users 200. Such sleep evaluation system 1 is capable of improving the appropriateness of a sleep evaluation.

Stated differently, sleep evaluation system 1 performs a method (sleep evaluation method) as shown in FIG. 6 through FIG. 8. The sleep evaluation method according to the present embodiment includes determining a criterion and evaluating. The determining of the criterion is determining an individual evaluation criterion on the basis of a sleep log that includes a log of sleep information relating to the sleep of specific user 100. The evaluating is performing an evaluation process of evaluating the sleep of specific user 100 from the specific sleep information, using both the individual evaluation criterion and a general evaluation criterion that is determined on the basis of sleep information relating to the sleep of a plurality of users 200. This aspect improves the appropriateness of a sleep evaluation. As with sleep evaluation stem 1, such sleep evaluation method is capable of improving the appropriateness of a sleep evaluation.

Sleep evaluation system 1 is implemented using a computer system. Stated differently, the method (sleep evaluation method) performed by sleep evaluation system 1 can be implemented by the computer system executing a program. Such program is a computer program for causing at least one processor to execute the sleep evaluation method. As with sleep evaluation system 1, such program is capable of improving the appropriateness of a sleep evaluation.

### (2) Variation

The embodiment of the present disclosure is not limited to the foregoing embodiment. The foregoing embodiment is capable of being modified in various manners in accordance with design, etc. so long as the aim of the present disclosure is achieved. The following recites variations of the foregoing embodiment. The variations described below can be applied in combination with each other as appropriate.

In one variation, obtainer 131 may obtain the specific sleep information directly from sensor 30. Stated differently, terminal device 20 does not necessarily have to obtain the specific sleep information from sensor 30. Meanwhile, the specific sleep information does not necessarily have to be obtained by sensor 30, and thus may be inputted by specific user 100 to terminal device 20 via input unit 21 of terminal device 20. In this case, obtainer 131 is simply required to obtain the specific sleep information from terminal device 20.

In another variation, obtainer 131 may obtain the sleep information of a plurality of users 200 in addition to the specific sleep information. Obtainer 131 may obtain items of sleep information from a plurality of terminal devices 20 to obtain a log of the sleep information of a plurality of users 200, and cause storage 12 to store the obtained log of the sleep information of a plurality of users 200. In this case, evaluation criterion determiner 132 may determine general evaluation criterion 123 on the basis of the log of the sleep information of a plurality of users 200.

In the foregoing embodiment, specific user 100 is included in a plurality of users 200, but does not necessarily have to be included in a plurality of users 200.

In another variation, evaluation criterion determiner 132 may update individual evaluation criterion 122 on a regular basis or at a predetermined timing. The predetermined timing can be, for example, the timing at which the number of days of specific sleep information included in sleep log 121 has increased as many as a specified number of days.

In another variation, sleep information does not have to include all of sleep duration, sleep efficiency, sleep latency (time taken to fall asleep), the number of mid-sleep awakenings, the state immediately before awakening, and the ratio of deep sleep, and may include sleep-related items different from these. The sleep information may include, for example, a time interval between timings at which mid-sleep awakenings occur.

In another variation, evaluation values do not have to be represented as points. Evaluation values are not limited to being represented as points, and thus may be represented by scale evaluation such as on a scale of one to five. The scale evaluation may be represented by grades or may be described quantitively.

In another variation, the weights in weight information 124 of the respective evaluation criteria of individual evaluation criterion 122 and general evaluation criterion 123 may be the same. Stated differently, ws1, ws2, ws3, ws4, ws5, and ws6 may be the same value. In this case, wg1, wg2, wg3, wg4, wg5, and wg6 are also the same value. Stated differently, using weight information 124, the weight may be assigned to the result of sleep evaluations (a total of evaluation values) that use individual evaluation criterion 122 and general evaluation criterion 123.

In another variation, weight information 124 is simply required to be information by which it is possible to assign weights to general evaluation criterion 123 and individual evaluation criterion 122. In the foregoing embodiment, general evaluation criterion 123 and individual evaluation criterion 122 each include evaluation conditions for determining evaluation values relating to sleep. Weight information 124 may be used to assign weights not to evaluation values but to evaluation conditions.

In another variation, upon determining weight information (goal weight information), weight determiner 133 may soon reflect such goal weight information on evaluations. Stated differently, the transition time over which the current weight information is changed to be the goal weight information do not necessarily have to be used.

In another variation, weight determiner 133 may change weight information 124 in accordance with a response from specific user 100 to the result of an evaluation process. For example, sleep evaluation system 1 may collect the level of satisfaction of specific user 100 with the result of an evaluation process to reflect the collected level of satisfaction on weight information 124. For example, for an item with which specific user 100 is unsatisfied among sleep duration, sleep efficiency, sleep latency (time taken to fall asleep), the number of mid-sleep awakenings, the state immediately before awakening, and the ratio of deep sleep, the corresponding weight of individual evaluation criterion 122 may be changed to be greater than that of general evaluation criterion 123. With this, it is possible to maintain a higher level of satisfaction of specific user 100 with the result of an evaluation process.

In another variation, weight determiner 133 may change weight information 124 in response to a reply from specific user 100 about sleep. For example, sleep evaluation system 1 may collect information about concerns of specific user 100 about sleep (points specific user 100 is worrying about) to reflect the collected information on weight information 124. For example, for an item specific user 100 is concerned about among sleep duration, sleep efficiency, sleep latency (time taken to fall asleep), the number of mid-sleep awakenings, the state immediately before awakening, and the ratio of deep sleep, the corresponding weight of individual evaluation criterion 122 may be changed to be greater than that of general evaluation criterion 123. With this, it is possible to maintain a higher level of satisfaction of specific user 100 with the result of an evaluation process.

In another variation, evaluator 134 does not have to obtain both result Vs of evaluating the sleep of specific user 100 using individual evaluation criterion 122 and result Vg of evaluating the sleep of specific user 100 using general evaluation criterion 123. Stated differently the first evaluation result does not have to include both result Vs of evaluating the sleep of specific user 100 using individual evaluation criterion 122 and result Vg of evaluating the sleep of specific user 100 using general evaluation criterion 123, and thus may include one of Vs and Vg. The result of an evaluation do not have to include the first evaluation result.

In another variation, evaluator 134 may reflect environment information relating to the environment in which specific user 100 sleeps on a sleep evaluation of specific user 100. The environment information can include a lighting environment, a temperature environment, a geographical environment, and a noise environment relating to the place in which specific user 100 sleeps. The use of the environment information improves the accuracy of an evaluation of the sleep of specific user 100.

In another variation, presenter 135 does not have to display the first evaluation result (first radar chart 141) and the second evaluation result (second radar chart 142) on the same screen as shown in FIG. 5. For example, presenter 135 may present the first evaluation result (first radar chart 141) and the second evaluation result (second radar chart 142) in a manner that the first evaluation result and the second evaluation result can be switched therebetween. Presenter 135 does not necessarily have to present a comparison of the first evaluation result and the second evaluation result. Presenter 135 may present the result of an evaluation not visually but in an auditory manner. Also, presenter 135 may only output data indicating the result of an evaluation process in a predetermined format.

In another variation, presenter 135 is simply required to present at least third region R13, and thus first region R11, second region R12, fourth region R14, and fifth region R15 are not essential. In particular, regarding fifth region R15, presenter 135 serves as the advice generator that generates an advice on the sleep of specific user 100. Without fifth region R15, presenter 135 does not have to serve as the advice generator.

Sleep evaluation system 1 according to the present disclosure includes, for example, a computer system. The computer system mainly includes a processor and a memory as hardware elements. The processor executes a program recorded in the memory of the computer system, thereby achieving the functions of sleep evaluation system 1 according to the present disclosure. The program may be preliminarily recorded in the memory of the computer system, provided via a telecommunications line, or recorded and provided in a non-transitory recording medium readable by the computer system, such as a memory card, an optical disc, and a hard disk drive. The processor of the computer system includes one or a plurality of electronic circuits including a semiconductor integrated circuit (IC) or a large-scale integration (LSI). The integrated circuits such as an IC and an LSI here are differently called depending on the degree of integration, and thus include integrated circuits known as a system LSI, a very large-scale integration (VLSI), or ultra large scale integration (ULSI). Also employable as the processor is a field-programmable gate array (FPGA) that is programmed after the manufacture of an LSI, or a logical device that allows for the reconfiguration of connections inside of an LSI or the reconfiguration of circuit partitions inside of the LSI. The plurality of electronic circuits may be integrated into a single chip or dispersedly provided in a plurality of chips. The plurality of chips may be integrated in a single device or dispersedly provided in a plurality of devices. The computer system here includes a microcontroller including at least one processor and at least one memory. The microcontroller thus also includes one or a plurality of electronic circuits including a semiconductor integrated circuit or a large-scale integration.

In the foregoing embodiment, sleep evaluation system 1 is implemented by server 10, but may be implemented by terminal device (information terminal) 20. For example, terminal device 20 may include evaluation criterion determiner 132 and evaluator 134. Stated differently, at least one processor included in a computer system that constitutes terminal device 20 executes a program, thereby serving as sleep evaluation system 1.

That a plurality of functions in sleep evaluation system 1 are integrated in a single cabinet is not essential to sleep evaluation system 1, and thus elements of sleep evaluation system 1 may be dispersedly provided in a plurality of cabinets. For example, sleep evaluation system 1 may be implemented by terminal device 20 and server 10. In this case, sleep evaluation system 1 may include: server 10 including evaluation criterion determiner 132 and evaluator 134; and terminal device 20 that is capable of communicating with server 10 and obtaining and presenting the result of an evaluation process performed by evaluator 134. Also, one or more of the functions of processing unit 13 can be implemented by server 10, and the other functions can be implemented by terminal device 20. For example, terminal device 20 may include obtainer 131 and presenter 135, and server 10 may include evaluation criterion determiner 132, weight determiner 133, and evaluator 134. Further, at least one of the functions of sleep evaluation system 1, e.g., one or more of the functions of processing unit 13 may be implemented by cloud (cloud computing), etc.

### (3) Aspects

As is obvious from the foregoing embodiment and variations, the present disclosure includes the aspects described below. To explicitly illustrate the correspondence with the embodiment, the reference marks are provided in brackets.

A first aspect is a sleep evaluation system (1) including an evaluation criterion determiner (132) and an evaluator (134). The evaluation criterion determiner (132) determines an individual evaluation criterion (122) on the basis of a sleep log (121) that includes a log of specific sleep information relating to the sleep of a specific user (100). The evaluator (134) performs an evaluation process of evaluating the sleep of the specific user (100) from the specific sleep information, using both the individual evaluation criterion (122) and the general evaluation criterion (123) that is determined on the basis of sleep information relating to the sleep of a plurality of users (200). This aspect improves the appropriateness of a sleep evaluation.

A second aspect is the sleep evaluation system (1) that is based on the first aspect. In the second aspect, the sleep evaluation system (1) further includes a weight determiner (133) that determines weight information (124) for each of the general evaluation criterion (123) and the individual evaluation criterion (122). The evaluator (134) performs the evaluation process, using the weight information (124). This aspect further improves the appropriateness of a sleep evaluation.

A third aspect is the sleep evaluation system (1) that is based on the second aspect. In the third aspect, the weight determiner (133) determines the weight information (124) on the basis of the result of comparing a first representative value indicating the result of evaluating the sleep of the specific user (100) using the general evaluation criterion (123) and a second representative value indicating the result of evaluating the sleep of a plurality of users (200) using the general evaluation criterion (123). This aspect further improves the appropriateness of a sleep evaluation.

A fourth aspect is the sleep evaluation system (1) that is based on the second or third aspect. In the fourth aspect, the general evaluation criterion (123) and the individual evaluation criterion (122) each include an evaluation condition for determining an evaluation value relating to sleep. The weight information (124) is information used to assign a weight to the evaluation value. This aspect further improves the appropriateness of a sleep evaluation.

A fifth aspect is the sleep evaluation system (1) that is based on any one of the second through fourth aspects. In the fifth aspect, the general evaluation criterion (123) and the individual evaluation criterion (122) each include an evaluation criterion for each of sleep-related items. The weight information (124) includes a weight of the evaluation criterion for each of the sleep-related items. This aspect further improves the appropriateness of a sleep evaluation.

A sixth aspect is the sleep evaluation system (1) that is based on any one of the second through fifth aspects. In the sixth aspect, the weight determiner (133) updates the weight information (124) when a predetermined condition is satisfied. This aspect further improves the appropriateness of a sleep evaluation.

A seventh aspect is the sleep evaluation system (1) that is based on any one of the second through sixth aspects. In the seventh aspect, after the weight information (124) is changed, the evaluator (134) performs the evaluation process again using the weight information (124) changed. This aspect further improves the appropriateness of a sleep evaluation.

An eighth aspect is the sleep evaluation system (1) that is based on any one of the second through seventh aspects. In the eighth aspect, the weight determiner (133) changes the weight information (124) in accordance with a response from the specific user (100) about the result of the evaluation process. This aspect further improves the appropriateness of a sleep evaluation.

A ninth aspect is the sleep evaluation system (1) that is based on any one of the first through eighth aspects. In the ninth aspect, the evaluator (134) evaluates the sleep of the specific user (100), using the general evaluation criterion (123) before the evaluation criterion determiner (132) determines the individual evaluation criterion (122). This aspect enables a sleep evaluation to be performed even before the individual evaluation criterion (122) is obtained.

A tenth aspect is the sleep evaluation system (1) that is based on any one of the first through ninth aspects. In the tenth aspect, the result of the evaluation process includes a first evaluation result indicating the result of evaluating the sleep of the specific user (100) using one of the individual evaluation criterion (122) and the general evaluation criterion (123) and a second evaluation result indicating the result of evaluating the sleep of the specific user (100) using both the individual evaluation criterion (122) and the general evaluation criterion (123). This aspect facilitates the grasp of a sleep evaluation.

An eleventh aspect is the sleep evaluation system (1) that is based on the tenth aspect. In the eleventh aspect, the sleep evaluation system (1) further includes a presenter (135) that presents a comparison of the first evaluation result and the second evaluation result. This aspect facilitates the grasp of a sleep evaluation.

A twelfth aspect is the sleep evaluation system (1) that is based on any one of the first through eleventh aspects. In the twelfth aspect, the sleep evaluation system (1) further includes a sensor (30) that obtains the specific sleep information. This aspect facilitates the obtainment of the specific sleep information.

A thirteenth aspect is the sleep evaluation system (1) that is based on any one of the first through twelfth aspects. In the thirteenth aspect, the evaluator (134) reflects environment information relating to the environment in which the specific user (100) sleeps on an evaluation relating to the sleep of the specific user (100). This aspect improves the accuracy of a sleep evaluation.

A fourteenth aspect is a sleep evaluation system (1) including a server (10) and a terminal device (20). The server (10) includes an evaluation criterion determiner (132) and an evaluator (134). The evaluation criterion determiner (132) determines an individual evaluation criterion (122) on the basis of a sleep log (121) that includes a log of specific sleep information relating to the sleep of a specific user (100). The evaluator (134) performs an evaluation process of evaluating the sleep of the specific user (100) from the specific sleep information, using both the individual evaluation criterion (122) and the general evaluation criterion (123) that is determined on the basis of sleep information relating to the sleep of a plurality of users (200). The terminal device (20) includes: a communicator (23) capable of communicating with the server (10); an output unit (22) that outputs information of the terminal device (20); and a processing unit (24) that controls the communicator (23) and the output unit (22). The processing unit (24) receives via the communicator (23) the result of the evaluation process from the evaluator (134) of the server (10) and outputs via the output unit (22) the result of the evaluation process. This aspect improves the appropriateness of a sleep evaluation.

A fifteenth aspect is a program for causing at least one processor of the processing unit (24) of the terminal device (20) in the sleep evaluation system (1) according to the fourteenth aspect to execute: receiving via the communicator (23) the result of the evaluation process from the evaluator (134) of the server (10); and outputting via the output unit (22) the result of the evaluation process. This aspect improves the accuracy of a sleep evaluation.

A sixteenth aspect is a sleep evaluation method including determining of a criterion and evaluating. The determining of a criterion is determining an individual evaluation criterion (122) on the basis of a sleep log (121) that includes a log of specific sleep information relating to the sleep of a specific user (100). The evaluating is performing an evaluation process of evaluating the sleep of the specific user (100) from the specific sleep information, using both the individual evaluation criterion (122) and the general evaluation criterion (123) that is determined on the basis of sleep information relating to the sleep of a plurality of users (200). This aspect improves the appropriateness of a sleep evaluation.

A seventeenth aspect is a program for causing at least one processor to execute the sleep evaluation method according to the sixteenth aspect. This aspect improves the appropriateness of a sleep evaluation.

An eighteenth aspect is a sleep evaluation method including: determining weight information (124) for each of an individual evaluation criterion (122) that is determined on the basis of a sleep log (121) that includes a log of specific sleep information relating to the sleep of a specific user (100) and a general evaluation criterion (123) that is determined on the basis of sleep information relating to the sleep of a plurality of users (200); and performing an evaluation process of evaluating the sleep of the specific user (100) from the specific sleep information, using the individual evaluation criterion (122), the general evaluation criterion (123), and the weight information (124). This aspect improves the appropriateness of a sleep evaluation.

A nineteenth aspect is the sleep evaluation method that is based on the eighteenth aspect. In the nineteenth aspect, the weight information (124) is changed on the basis of the number of days of accumulation for which the specific sleep information has been accumulated, or a response from the specific user (100) about the result of the evaluation process, and the evaluation process is performed using the weight information (124) changed. This aspect further improves the appropriateness of a sleep evaluation by changing the weight information.

A twentieth aspect is the sleep evaluation method that is based on the nineteenth aspect. In the twentieth aspect, the weight information (124) is changed to increase the ratio of the weight of the individual evaluation criterion (122) to a total of the weight of the individual evaluation criterion (122) and the weight of the general evaluation criterion (123) with an increase in the number of days of accumulation. This aspect, in which the ratio of the weight of individual evaluation criterion (122) is gradually increased with an increase in the number of days of accumulation, further improves the appropriateness of a sleep evaluation.

A twenty-first aspect is the sleep evaluation method that is based on the twentieth aspect. In the twenty-first aspect, an increase rate of the ratio to the increase in the number of days of accumulation is changed based on the result of comparing a first representative value indicating the result of evaluating the sleep of the specific user (100) using the general evaluation criterion (123) and a second representative value indicating the result of evaluating the sleep of the plurality of users (200) using the general evaluation criterion (123); and the weight information (124) is changed based on the increase rate changed. This aspect, in which the increase rate of the ratio is changed on the basis of the result of comparing the first representative value and the second representative value, facilitates an appropriate change of the increase rate of the ratio in accordance with the sleep state of the specific user (100), thus further improving the appropriateness of a sleep evaluation.

A twenty-second aspect is the sleep evaluation method that is based on the twenty-first aspect. In the twenty-second aspect, the increase rate is increased when the first representative value is worse than the second representative value, and the increase rate is decreased when the first representative value is better than the second representative value. This aspect further facilitates an appropriate change of the increase rate of the ratio in accordance with the sleep state of the specific user (100), thus further improving the appropriateness of a sleep evaluation.

A twenty-third aspect is the sleep evaluation method that is based on the nineteenth aspect. In the twenty-third aspect, the result of evaluating the sleep of the specific user (100) using the individual evaluation criterion (122) and the result of evaluating the sleep of the specific user (100) using the general evaluation criterion (123) are presented to the specific user (100) and a response from the specific user (100) is received. The weight of the individual evaluation criterion (122) is increased to be greater than the weight of the general evaluation criterion (123), when the specific user (100) selects the result of evaluating the sleep of the specific user (100) using the individual evaluation criterion (122), and the weight of the individual evaluation criterion (122) is decreased to be smaller than the weight of the general evaluation criterion (123), when the specific user (100) selects the result of evaluating the sleep of the specific user (100) using the general evaluation criterion (123). This aspect facilitates an appropriate setting of the weight of the individual evaluation criterion (122) and the weight of the general evaluation criterion (123) in accordance with the response from the specific user (100), thus further improving the appropriateness of a sleep evaluation.

A twenty-fourth aspect is the sleep evaluation method that is based on any one of the eighteenth through twenty-third aspects. In the twenty-fourth aspect, the general evaluation criterion (123) and the individual evaluation criterion (122) each include an evaluation condition for determining an evaluation value relating to sleep, and the weight information (124) is information used to assign a weight to the evaluation value. This aspect further improves the appropriateness of a sleep evaluation.

A twenty-fifth aspect is the sleep evaluation method that is based on any one of the eighteenth through twenty-fourth aspects. In the twenty-fifth aspect, the general evaluation criterion (123) and the individual evaluation criterion (122) each include an evaluation criterion for each of sleep-related items; and the weight information (124) includes a weight of the evaluation criterion for each of the sleep-related items. This aspect further improves the appropriateness of a sleep evaluation.

A twenty-sixth aspect is the sleep evaluation method that is based on any one of the eighteenth through twenty-fifth aspects. In the twenty-sixth aspect, the weight information (124) is updated when a predetermined condition is satisfied. This aspect further improves the appropriateness of a sleep evaluation.

A twenty-seventh aspect is the sleep evaluation method that is based on any one of the eighteenth through twenty-sixth aspects. In the twenty-seventh aspect, the result of the evaluation process includes: a first evaluation result indicating the result of evaluating the sleep of the specific user (100) using one of the individual evaluation criterion (122) and the general evaluation criterion (123); and a second evaluation result indicating the result of evaluating the sleep of the specific user (100) using both the individual evaluation criterion (122) and the general evaluation criterion (123). This aspect facilitates the grasp of a sleep evaluation.

A twenty-eighth aspect is the sleep evaluation method that is based on the twenty-seventh aspect. In the twenty-eighth aspect, a comparison of the first evaluation result and the second evaluation result is presented. This aspect facilitates the grasp of a sleep evaluation.

A twenty-ninth aspect is a sleep evaluation system (1) including: a weight determiner (133) that determines weight information (124) for each of an individual evaluation criterion (122) that is determined on the basis of a sleep log (121) that includes a log of specific sleep information relating to sleep of a specific user (100) and a general evaluation criterion (123) that is determined on the basis of sleep information relating to sleep of a plurality of users (200); an evaluator (134) that performs an evaluation process of evaluating the sleep of the specific user (100) from the specific sleep information, using the individual evaluation criterion (122), the general evaluation criterion (123), and the weight information (124). This aspect improves the appropriateness of a sleep evaluation.

A thirtieth aspect is a sleep evaluation system (1) that includes a server (10) and a terminal device (20) capable of communicating with the server (10). The server (10) includes: a weight determiner (133) that determines weight information (124) for each of an individual evaluation criterion (122) that is determined on the basis of a sleep log (121) that includes a log of specific sleep information relating to sleep of a specific user (100) and a general evaluation criterion (123) that is determined on the basis of sleep information relating to sleep of a plurality of users (200); and an evaluator (134) that performs an evaluation process of evaluating the sleep of the specific user (100) from the specific sleep information, using the individual evaluation criterion (122), the general evaluation criterion (123), and the weight information (124). The terminal device (20) includes: a communicator (23) capable of communicating with the server (10); an output unit (22) that outputs information of the terminal device (20); and a processing unit (24) that controls the communicator (23) and the output unit (22). The processing unit (24) receives via the communicator (23) the result of the evaluation process from the evaluator (134) of the server (10), and outputs via the output unit (22) the result of the evaluation process. This aspect improves the appropriateness of a sleep evaluation.

A thirty-first aspect is a terminal device (20) capable of communicating with a server (10). The server (10) includes: a weight determiner (133) that determines weight information (124) for each of an individual evaluation criterion (122) that is determined on the basis of a sleep log (121) that includes a log of specific sleep information relating to sleep of a specific user (100) and a general evaluation criterion (123) that is determined on the basis of sleep information relating to sleep of a plurality of users (200); an evaluator (134) that performs an evaluation process of evaluating the sleep of the specific user (100) from the specific sleep information, using the individual evaluation criterion (122), the general evaluation criterion (123), and the weight information (124); a communicator (23) capable of communicating with the server (10); an output unit (22) that outputs information of the terminal device (20); and a processing unit (24) that controls the communicator (23) and the output unit (22). The processing unit (24) receives via the communicator (23) the result of the evaluation process from the evaluator (134) of the server (10), and outputs via the output unit (22) the result of the evaluation process. This aspect improves the appropriateness of a sleep evaluation.

A thirty-second aspect is a program for causing at least one processor to execute a sleep evaluation method that includes: determining weight information (124) for each of an individual evaluation criterion (122) that is determined on the basis of a sleep log (121) that includes a log of specific sleep information relating to sleep of a specific user (100) and a general evaluation criterion (123) that is determined on the basis of sleep information relating to sleep of a plurality of users (200); and performing an evaluation process of evaluating the sleep of the specific user (100) from the specific sleep information, using the individual evaluation criterion (122), the general evaluation criterion (123), and the weight information (124). This aspect improves the appropriateness of a sleep evaluation.

A thirty-third aspect is a program for causing at least one processor in a processing unit (24) of a terminal device (20) in a sleep evaluation system (1) that includes a server (10) and a terminal device (20) capable of communicating with the server (10). The server (10) includes: a weight determiner (133) that determines weight information (124) for each of an individual evaluation criterion (122) that is determined on the basis of a sleep log (121) that includes a log of specific sleep information relating to sleep of a specific user (100) and a general evaluation criterion (123) that is determined on the basis of sleep information relating to sleep of a plurality of users (200); and an evaluator (134) that performs an evaluation process of evaluating the sleep of the specific user (100) from the specific sleep information, using the individual evaluation criterion (122), the general evaluation criterion (123), and the weight information (124). The terminal device (20) includes: a communicator (23) capable of communicating with the server (10); an output unit (22) that outputs information of the terminal device (20); and the processing unit (24) that controls the communicator (23) and the output unit (22). The processing unit (24) receives via the communicator (23) the result of the evaluation process from the evaluator (134) of the server (10), and outputs via the output unit (22) the result of the evaluation process. The program causes the at least one processor to execute: receiving via the communicator (23) the result of the evaluation process from the evaluator (134) of the server (10); and outputting via the output unit (22) the result of the evaluation process. This aspect improves the appropriateness of a sleep evaluation.

Note that the second through the thirteenth aspects can be modified as appropriate to be applied to the sixteenth and the seventeenth aspects.

1. A sleep evaluation method comprising:
determining weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
performing an evaluation process of evaluating the sleep of the specific user from the specific sleep information, using the individual evaluation criterion, the general evaluation criterion, and the weight information.

2. The sleep evaluation method according to claim 1,
wherein the weight information is changed based on a total number of days of accumulation for which the specific sleep information has been accumulated or a response from the specific user about a result of the evaluation process, and
the evaluation process is performed, using the weight information changed.

3. The sleep evaluation method according to claim 2,
wherein the weight information is changed to increase a ratio with an increase in the total number of days of accumulation, the ratio being a ratio of a weight of the individual evaluation criterion to a total of the weight of the individual evaluation criterion and a weight of the general evaluation criterion.

4. The sleep evaluation method according to claim 3,
wherein an increase rate of the ratio to the increase in the total number of days of accumulation is changed based on a result of comparing a first representative value and a second representative value, the first representative value indicating a result of evaluating the sleep of the specific user using the general evaluation criterion, the second representative value indicating a result of evaluating the sleep of the plurality of users using the general evaluation criterion, and
the weight information is changed based on the increase rate changed.

5. The sleep evaluation method according to claim 4,
wherein the increase rate is increased when the first representative value is worse than the second representative value, and
the increase rate is decreased when the first representative value is better than the second representative value.

6. The sleep evaluation method according to claim 2, further comprising:
presenting, to the specific user, a result of evaluating the sleep of the specific user using the individual evaluation criterion and a result of evaluating the sleep of the specific user using the general evaluation criterion, and receiving a response from the specific user;
increasing a weight of the individual evaluation criterion to be greater than a weight of the general evaluation criterion, when the specific user selects the result of evaluating the sleep of the specific user using the individual evaluation criterion; and
decreasing the weight of the individual evaluation criterion to be smaller than the weight of the general evaluation criterion, when the specific user selects the result of evaluating the sleep of the specific user using the general evaluation criterion.

7. The sleep evaluation method according to claim 1,
wherein the general evaluation criterion and the individual evaluation criterion each include an evaluation condition for determining an evaluation value relating to sleep, and
the weight information is information used to assign a weight to the evaluation value.

8. The sleep evaluation method according to claim 1,
wherein the general evaluation criterion and the individual evaluation criterion each include an evaluation criterion for each of sleep-related items, and
the weight information includes a weight of the evaluation criterion for each of the sleep-related items.

9. The sleep evaluation method according to claim 1, further comprising:
updating the weight information when a predetermined condition is satisfied.

10. The sleep evaluation method according to claim 1,
wherein a result of the evaluation process includes:
a first evaluation result indicating a result of evaluating the sleep of the specific user using one of the individual evaluation criterion and the general evaluation criterion; and
a second evaluation result indicating a result of evaluating the sleep of the specific user using both the individual evaluation criterion and the general evaluation criterion.

11. The sleep evaluation method according to claim 10, further comprising:
presenting a comparison of the first evaluation result and the second evaluation result.

12. A sleep evaluation system comprising:
a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
an evaluator that performs an evaluation process of evaluating the sleep of the specific user from the specific sleep information, using the individual evaluation criterion, the general evaluation criterion, and the weight information.

13. A sleep evaluation system comprising:
a server; and
a terminal device capable of communicating with the server,
wherein the server includes:
a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
an evaluator that performs an evaluation process of evaluating the sleep of the specific user from the specific sleep information, using the individual evaluation criterion, the general evaluation criterion, and the weight information, and
the terminal device includes:
a communicator capable of communicating with the server;
an output unit that outputs information of the terminal device; and
a processing unit that controls the communicator and the output unit, and
the processing unit receives via the communicator a result of the evaluation process from the evaluator of the server, and outputs via the output unit the result of the evaluation process.

14. A terminal device capable of communicating with a server that includes:
a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
an evaluator that performs an evaluation process of evaluating the sleep of the specific user from the specific sleep information, using the individual evaluation criterion, the general evaluation criterion, and the weight information, and
the terminal device comprising:
a communicator capable of communicating with the server;
an output unit that outputs information of the terminal device; and
a processing unit that controls the communicator and the output unit,
wherein the processing unit receives via the communicator a result of the evaluation process from the evaluator of the server, and outputs via the output unit the result of the evaluation process.

15. A non-transitory computer-readable recording medium having a program recorded thereon for causing at least one processor to execute a sleep evaluation method that includes:
determining weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
performing an evaluation process of evaluating the sleep of the specific user from the specific sleep information, using the individual evaluation criterion, the general evaluation criterion, and the weight information.

16. A non-transitory computer-readable recording medium having a program recorded thereon for causing at least one processor in a processing unit of a terminal device in a sleep evaluation system that includes:
a server; and
a terminal device capable of communicating with the server,
wherein the server includes:
a weight determiner that determines weight information for each of an individual evaluation criterion and a general evaluation criterion, the individual evaluation criterion being determined based on a sleep log that includes a log of specific sleep information relating to sleep of a specific user, the general evaluation criterion being determined based on sleep information relating to sleep of a plurality of users; and
an evaluator that performs an evaluation process of evaluating the sleep of the specific user from the specific sleep information, using the individual evaluation criterion, the general evaluation criterion, and the weight information, and
the terminal device includes:
a communicator capable of communicating with the server;
an output unit that outputs information of the terminal device; and
the processing unit that controls the communicator and the output unit, and
the processing unit receives via the communicator a result of the evaluation process from the evaluator of the server, and outputs via the output unit the result of the evaluation process, and
the program causes the at least one processor to execute:
receiving via the communicator the result of the evaluation process from the evaluator of the server; and
outputting via the output unit the result of the evaluation process.
